(19) United States
(12) Patent Application Publication
(10) Pub. No.: US 2019/0307390 A1
(43) Pub. Date: Oct. 10, 2019

(54) DEVICE AND METHOD FOR DETECTING DISEASE STATES ASSOCIATED WITH LIPOPIGMENTS

(71) Applicants: Massachusetts Institute of Technology, Cambridge, MA (US); The General Hospital Corporation, Boston, MA (US)

(72) Inventors: Moungi G. Bawendi, Cambridge, MA (US); Rakesh Jain, Wellesley, MA (US); Oliver Thomas Bruns, Garching (DE); Jessica Ann Carr, Cambridge, MA (US); Klaus van Leyen, Medford, MA (US); Wilhelmus Kwanten, Wilrijk (BE); Ivy Xiaoyu Chen, Brookline, MA (US)

(73) Assignees: Massachusetts Institute of Technology, Cambridge, MA (US); The General Hospital Corporation, Boston, MA (US)

(21) Appl. No.: 16/379,290

(22) Filed: Apr. 9, 2019

Related U.S. Application Data
(60) Provisional application No. 62/654,665, filed on Apr. 9, 2018.

(51) Int. Cl. A61B 5/00; G01J 3/28; G01J 3/44

(52) U.S. Cl. CPC A61B 5/4244; A61B 5/0071; A61B 5/4842; A61B 2503/40; G01J 3/2823; G01J 3/4406; A61B 5/742

(57) ABSTRACT

Systems and methods for measuring autofluorescent signals from lipopigments associated with various disease states are disclosed.

### CROSS-REFERENCE TO RELATED APPLICATIONS

This application claims the benefit under 35 U.S.C. § 119(e) of U.S. provisional application Ser. No. 62/654,665, filed Apr. 9, 2018, the disclosure of which is incorporated by reference in its entirety.

### GOVERNMENT LICENSE RIGHTS

This invention was made with Government support under Grant Nos. CA080124 and EB015871 awarded by the National Health Institutes (NIH) and Grant No. W911NF-13-D-0001 awarded by the Army Research Office (ARO). The Government has certain rights in the invention.

### TECHNICAL FIELD

Systems and methods for detecting disease states associated with cumulative lipopigments are disclosed.

### BACKGROUND

Current in vivo imaging technologies fail to provide high resolution, desirable penetration depths, and sensitivity simultaneously, which limits their widespread adoption for identifying diseases. For example, high resolution and high sensitivity imaging is straightforward on single cells using visible light imaging techniques. However, when imaging whole animals and their tissues, resolution and sensitivity of subsurface tissue features are drastically reduced due to scattering and absorption of light by surrounding tissue. Another major limitation of conventional in vivo imaging technologies is the intense background autofluorescence of tissue at the same wavelengths as the fluorescence emission wavelengths used to detect various conditions. This overlap of autofluorescence emission wavelengths may inhibit disease detection. In one such example, traditional fluorescence imaging with visible and near infrared wavelengths suffers from poor contrast against the background autofluorescence signals from normal cells and tissues.

### SUMMARY

In some embodiments, methods and systems related to autofluorescence based imaging in the near infrared (NIR) and shortwave infrared (SWIR) spectral regions are described.

In certain embodiments, a method includes exposing tissue that comprises lipopigments when in a diseased state to an excitation source. The lipopigments may have at least a portion of an autofluorescence spectrum at wavelengths between 700 nm and 1200 nm. The method may also include imaging the tissue at one or more wavelengths between 700 nm and 1200 nm.

According to certain embodiments, a method includes exposing tissue that comprises lipopigments when in a diseased state to an excitation source. The lipopigments may have at least a portion of an autofluorescence spectrum at wavelengths between 700 nm and 2000 nm. Further, the diseased state may include at least one of non-alcoholic fatty liver disease and/or lysosomal storage diseases. The method may also include imaging the tissue at one or more wavelengths between 700 nm and 2000 nm, and comparing the imaged tissue to an intensity threshold to determine if the tissue is in the diseased state of non-alcoholic fatty liver disease and/or lysosomal storage diseases.

In certain embodiments, a medical imaging device may include an excitation source that emits excitation light at one or more wavelengths between 400 nm and 850 nm, a detector configured to detect an autofluorescence signal emitted from lipopigments from tissue being imaged with the device with wavelengths between or equal to 700 nm and 1200 nm, and a computing device that receives the detected autofluorescence signal from the detector. The computing device may be configured to continuously determine a disease state of the tissue being imaged in real time.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1A is, according to some embodiments, a graph of an excitation wavelength and a corresponding emission wavelength;

FIG. 1B is, in accordance with certain embodiments, a graph of an excitation wavelength for a multi-photon excitation source and a corresponding emission wavelength;

FIG. 2 is a schematic representation of an excitation unit, transmission unit, and corresponding detection unit according to one embodiment;

FIG. 3 is a schematic representation of an excitation unit, transmission unit, and corresponding detection unit according to one embodiment;

FIG. 4 is an exemplary flow diagram of a method for tracking the progression and/or regression of a disease over time;

FIG. 5A is a near infrared/shortwave infrared autofluorescence image of the liver and the genitourinary anatomy detected in vivo in a non-alcoholic fatty liver disease mouse model;

FIG. 5B is a near infrared/shortwave infrared autofluorescence image of the excised liver tissue of the mouse model shown in FIG. 5A;

FIG. 5C is a near infrared/shortwave infrared autofluorescence image of cells in liver tissue from a liver cirrhosis mouse model;

FIG. 6A is an autofluorescence image of cirrhotic liver tissue without a Sudan Black B stain using a Cy5 excitation and emission channel;

FIG. 6B is an autofluorescence image of the cirrhotic liver tissue shown in FIG. 6A without a Sudan Black B stain using a near infrared/shortwave infrared channel;

FIG. 7A is an autofluorescence image of cirrhotic liver tissue with a Sudan Black B stain using a Cy5 channel;

FIG. 7B is an autofluorescence image of the cirrhotic liver tissue shown in FIG. 7A with a Sudan Black B stain using a near infrared/shortwave infrared channel;

FIG. 8 is a diagram of exemplary excitation and emission wavelengths for microscope filter cube settings;

FIG. 9 is an absorption spectrum of Sudan Black B;

FIG. 10A is an image of Sudan Black B-stained cirrhotic liver tissue (paraformaldehyde-fixed and paraffin embedded) from a mouse model of cirrhosis using visible illumination and detection, indicating areas of lipofuscin/ceroid in black;

FIG. 10B is an image of the same tissue slice shown in FIG. 10A showing NIR/SWIR autofluorescence of the Sudan Black B-positive areas associated with lipofuscin/ceroid;

FIG. 11A is an autofluorescence image of a mouse model fed a control diet (CD) for 12 weeks which does not induce non-alcoholic fatty liver disease;

FIG. 11B is an autofluorescence image of a mouse model fed choline-deficient, L-amino acid-defined, high-fat diet (CDAHFD) for 12 weeks to induce non-alcoholic fatty liver disease;

FIG. 12 shows representative images of the ex vivo imaged livers of mice models on respectively a CD and CDAHFD for 3 weeks, 6 weeks, 9 weeks, and 12 weeks;

FIG. 13A is a graph showing the quantification (in counts per millisecond) of near infrared/shortwave infrared autofluorescence in the ex vivo livers of mice on the CD compared to mice on the CDAHFD for the indicated period; and

FIG. 13B is a graph quantifying ex vivo liver intensity represented by the percent increase in signal versus the average intensity of the controls at the respective time point;

FIG. 14 is a graph of autofluorescence intensity measured during noninvasive in vivo imaging of CCl₄ induced fibrosis in rodent liver (CCl₄/OO) compared to olive oil (OO) only controls and no treatment controls;

FIG. 15 is a graph of autofluorescence intensity measured during ex vivo imaging of CCl₄ induced fibrosis in rodent liver;

FIG. 16 is a graph comparing noninvasive in vivo liver autofluorescence intensity between healthy mice with no treatment (SC) and fibrotic mice four weeks after common bile duct ligation (CBDL);

FIG. 17 is a graph comparing ex vivo liver autofluorescence intensity between healthy mice with no treatment (SC) and fibrotic mice four weeks after common bile duct ligation (CBDL);

FIG. 18 is a graph of percent area of measured autofluorescence observed in liver tissue slices from control and CCl₄ treated mice using autofluorescence microscopy;

FIG. 19A is a composite image of brightfield and autofluorescence images of 5 μm slices of F4/80 stained liver tissue from mice with CCl₄ induced fibrosis;

FIG. 19B is a composite image of brightfield and autofluorescence images of 5 μm slices of Sudan Black B stained liver tissue from mice with CCl₄ induced fibrosis;

FIG. 19C is a composite image of brightfield and autofluorescence images of 5 μm slices of Nile Blue Sulfate stained liver tissue from mice with CCl₄ induced fibrosis;

FIG. 19D is a composite image of brightfield and autofluorescence images of 5 μm slices of PAS-D stained liver tissue from mice with CCl₄ induced fibrosis;

FIG. 19E is a composite image of brightfield and autofluorescence images of 5 μm slices of PAS-D stained liver tissue from control mice;

FIG. 20 is a schematic of the treatment and testing methodology used for imaging of regression of CCl₄ induced fibrosis;

FIG. 21 is a graph comparing noninvasive in vivo measured autofluorescence intensities between healthy tissue, fibrotic tissue, and fibrotic tissue that has undergone regression for CCl₄ induced fibrosis;

FIG. 22 is a graph comparing ex vivo measured autofluorescence intensities between healthy tissue, fibrotic tissue, and fibrotic tissue that has undergone regression for CCl₄ induced fibrosis;

FIG. 23 is a graph comparing percent area of measured autofluorescence in liver tissue slices from healthy tissue, fibrotic tissue, and fibrotic tissue that has undergone regression for CCl₄ induced fibrosis; and

FIG. 24 is a graph of in vivo and ex vivo measured autofluorescence signals for homozygous Mcoln1 knock-out mouse model of mucolipidosis type IV as compared to controls for different organs.

### DETAILED DESCRIPTION

The Inventors have recognized the benefits associated with autofluorescence based imaging in the near infrared (NIR) and shortwave infrared (SWIR) spectral regions to improve the contrast and detection of various disease states. Specifically, and without wishing to be bound by theory, the longer imaging wavelength of the NIR/SWIR spectrum may reduce the photon scattering processes, thus maximizing transmission of the imaged light through a material (e.g., tissue) within the NIR/SWIR spectrum. Furthermore, unlike the visible region, the NIR/SWIR regime contains very little background autofluorescence from healthy tissues, especially in skin and muscle. This reduced background autofluorescence signal may improve the contrast with the corresponding autofluorescence signal from diseased tissue.

In addition to the above, the Inventors have recognized that the autofluorescence signals detected from various observed diseased tissues may originate from autofluorescence from accumulated lipopigments. Specifically, the Inventors have observed autofluorescence signals in the NIR/SWIR wavelength ranges from lipopigments including, for example, non-degradable oxidized intracellular material such as lipofuscin, ceroid, or lipofuscin-like lipopigments. These lipopigments may be associated with specific disease states as detailed further below. In view of the above, the Inventors have found that it is possible to optimize autofluorescence imaging methods and systems to accurately detect disease states that are associated with the accumulation and/or presence of these lipopigments. Thus, the currently disclosed systems and methods may improve the ability to easily distinguish between pathological and non-pathological biological structures.

Without wishing to be bound by theory, accumulation of lipopigments (such as oxidized intracellular materials including lipofuscin, ceroid, and/or lipofuscin-like lipopigments) in tissue may occur when autophagy is increased. For example, in some cases, the accumulation of lipopigments occurs as a result of an increase of delivered substrates (e.g., molecules, organelles, etc.) to lysosomes, such that the lysosomes cannot withstand the increased delivery of substrates, resulting in an increase in the oxidation and/or cross-linking of otherwise partially degradable material (e.g., an increase in lipofuscin, ceroid, and/or lipofuscin-like lipopigments). Consequently, in some cases, an increase in autophagy and related increase in lipofuscin, ceroid, and/or lipofuscin-like lipopigments may lead to an increase in cellular stress, decreased energy production, and ultimately, cell death. Due to the increased autofluorescence of these lipopigments, disease states affiliated with changes in lipopigment quantity may be detected by changes in the corresponding detected autofluorescence signal as detailed below. For example, the accumulation of intracellular oxidized material, such as lipofuscin, ceroid, and/or lipofuscin-like lipopigments, can be found in the central nervous system or liver tissue as a consequence of oxidative stress, as a response to pathological conditions, or in response to the presence of toxic compounds. Lipofuscin, ceroid, and/or lipofuscin-like lipopigments may also be associated with intracellular degradation pathways that may be linked to neurodegenerative diseases including Huntington's, Parkinson's, and Alzheimer's disease. As detailed further below, accumulation of lipopigments may also be associated with lysosomal storage diseases, which are inborn errors of metabolism, like the neuronal ceroid lipofuscinosis family of neurodegenerative diseases.

In view of the above, the systems and methods described herein may include autofluorescence imaging of tissues that exhibit increased amounts of lipopigments when in a diseased state. Accordingly, due to the lipopigments exhibiting autofluorescence signals, changes in a detected autofluorescence intensity may be correlated with an associated disease state. Furthermore, according to certain embodiments, the lipopigments may exhibit autofluorescence signals that are correlated with the cumulative amount of an associated disease state (e.g., in a tissue) that are due to lipopigments. For example, the amount of lipopigments present in the tissue may be directly correlated to the severity and/or extent of the associated disease state in the tissue.

In certain embodiments, the lipopigments discussed herein may generally be understood as granules consisting of an autofluorescent pigment and lipid components enclosed by a common, continuous membrane. In some cases, the lipopigments may be a lipid oxidation product, which may also be known as oxidized intracellular material. In some cases, lipopigments may be symptomatic of membrane, mitochondria, or lysosome damage, as well as the break-down of the intracellular pathway macro-autophagy. According to some embodiments, lipopigments may comprise protein residues and/or lipids.

According to some embodiments, the term lipopigment may refer to relatively non-degradable oxidized intracellular materials such as lipofuscin, ceroid, and/or lipofuscin-like lipopigments. Lipofuscin, in some cases, may be referred to as an “aging pigment” and/or “wear and tear pigment”, as it has long been associated with studies of senescence. Lipofuscin may be found in post-mitotic cells undergoing regressive changes (e.g., in neurons, cardiac myocytes, the liver, and retinal pigment epithelial cells). Ceroid may generally be understood as corresponding to a broad category of lipofuscin-like pigments unassociated with aging, but instead with disease. Ceroid can be found in a variety of cells, including, but not limited to, cells in the brain, liver, pancreas, testis, seminal vesicles, and the prostate. The presence of ceroids can be used as a diagnostic parameter in a variety of pathological conditions such as, but not limited to, tumors (e.g., prostatic adenocarcinoma, and/or carcinoma of the palate), atherosclerosis, malnutrition, toxic injury, and retinal degeneration.

As would be generally understood by those of ordinary skill in the art, as used herein, the terms lipopigment, lipofuscin, ceroid, and/or other similar terms may sometimes be used interchangeably. For instance, these terms are oftentimes used interchangeably to refer to the same cellular substance with similar physio-chemical and histochemical properties. However, some may distinguish the ceroids from lipofuscin based on the underlying etio-pathophysiological mechanisms.

In view of the above, disease states which may be associated with increased and/or changed amounts of lipopigments (e.g., ceroids and/or lipofuscin) may be detected based on correspondingly increased and/or changed autofluorescence signal intensities. Appropriate disease states that may be detected include, but are not limited to: cancerous or cancer-related tissues; cirrhotic tissues; fibrotic and scarred tissue; radiation-treated tissues; inflamed tissues; senescence (e.g., physiologically aged tissue or pathophysiologically aged tissue); stressed tissues; atherosclerotic lesions and plaques in vessels or tissues; neurodegenerative disease affected tissues, e.g. Alzheimer's disease, Alzheimer's Plaques, Parkinson's disease; angiography, including angiography of the eye and the following specific applications such as acute posterior multi-focal placoid pigment epitheliopathy, exudative senile macular degeneration, hemorrhagic detachment of retinal pigment epithelium, retinal hemorrhage, retinal neovascularization, serous detachment of retinal pigment epithelium, Behcet's disease (Behcet's syndrome), choroidal melanoma; critical limb ischemia, diabetic macular edema, Drusen differentiation, macular schisis, parasagittal meningioma, prediction of post-operative thrombosis in the internal jugular vein, prediction of wound complications in ventral hernia repair, sarcoidosis, scleritis and posterior scleritis, sentinel lymph node mapping, spinal dural arteriovenous fistula, Vogt-Koyanagi-Harada disease, and other appropriate uses for angiography; fibrosis or cirrhosis of the liver resulting from necro-inflammation (e.g. consequent to hepatotoxicity); as well as other applicable diseased, abnormal, or other tissue states of interest.

Exclusive of the above-noted disease states, the currently disclosed systems and methods may also be used to detect one or more of the following disease states. For example, autofluorescence of lipopigments may be used to detect non-alcoholic fatty liver disease. Accumulation of lipopigments may also be associated with lysosomal storage diseases and lysosomal related organelles, which are inborn errors of metabolism (e.g., Chediak Higashi syndrome, Hermansky-Pudlak syndrome, Griscelli syndrome, and Wiskott-Aldrich Syndrome). In certain embodiments, accumulation of lipopigments may be associated with the neuronal ceroid lipofuscinosis family of neurodegenerative diseases (e.g., Batten's Disease). Other disease states include sphingolipidoses, Tay-Sachs disease, Niemann-Pick disease, metachromatic leukodystrophy, mucopolysaccharidoses (e.g., Sanfilippo syndrome), progeria (also known as Hutchinson-Gilford disease), viral hepatitis, necro-inflammatory diseases of the liver, malnutrition, specific nutritional deficiencies (e.g., choline deficiency, vitamin E deficiency, vitamin D deficiency). Certain other disease states that may be associated with lipopigments include toxicities due to inhibitors of lysosomal enzymes, hormone administration, drug-induced side effects, and/or chronic alcohol consumption. In some cases, the accumulation of lipopigments may be associated with disease states such as atherosclerosis (e.g., stroke and/or infarct causing plaques), oxidative stress (e.g., hyperoxia, hypoxia), muscular dystrophy (e.g., Duchenne via oxidative stress), hemorrhage or infarct zones of the brain, and/or diseases related with increased autophagy (e.g., as observed in the 12/15-lipoxygenase knock out model).

Methods for detecting the presence of lipopigments using NIR/SWIR autofluorescence-based techniques may be attractive because information regarding the disease state of the tissue (e.g., of an animal model) can be acquired in real-time. Autofluorescence techniques also do not require sample processing, as the detected signal is an intrinsic property of the tissue. Accordingly, in some embodiments, the methods described herein may be implemented as nondestructive techniques for real-time, live cell analysis, and can be performed in vivo without the need for sample excision. Thus, the methods and systems may be used for imaging of any appropriate subject including human patients and/or animal models. These methods may lead to a better understanding of which disease states can be detected using autofluorescence from the presence of lipopigments which may include oxidized intracellular material such as lipofuscin, ceroids, and/or other similar lipofuscin-like pigments as well as improved systems and methods of operation. Additionally, the methods and systems described herein can be used to distinguish between abnormal or diseased and healthy tissue. Thus, the systems and methods described herein may accelerate investigations into disease mechanisms and/or serve as diagnostic or prognostic medical techniques and tools. Of course, while methods for detecting disease states without sample excision are described, embodiments in which imaging is done during surgical procedures and/or on excised tissue are also contemplated as the disclosure is not so limited.

As noted above, in some embodiments, the lipopigments that are being imaged may exhibit a peak autofluorescence and absorption spectrum that is at least partially outside of the NIR/SWIR spectrum. However, the autofluorescent spectrum of these lipopigments may include a tail that at least partially extends into the NIR/SWIR spectrum. In such an embodiment, a corresponding imaging or diagnostic device may include an excitation source that emits electromagnetic radiation within the absorption spectrum of the lipopigments and outside of the NIR/SWIR spectrum. The device may also include a detector that detects fluorescence emitted from the fluorescent component within the tail portion of the spectrum located in the NIR/SWIR spectrum. In certain embodiments, it is possible to excite within the visible spectrum and detect in the NIR/SWIR spectrum. In some cases, it is possible to excite within the NIR spectrum and detect in the NIR/SWIR spectrum.

In another embodiment, the inventors have recognized that imaging of the lipopigments in a particular spectrum, such as the NIR/SWIR spectrum, may reduce, and/or substantially eliminate, other autofluorescence signals associated with surrounding tissue. Therefore, a ratio of an intensity of the autofluorescence spectrum of the lipopigments and a corresponding intensity of the autofluorescence of surrounding tissue may be greater in the tail portion of the autofluorescence spectrum than at a peak fluorescent emission wavelength of the lipopigments. Consequently, in such an embodiment, a corresponding device may include an excitation source that emits electromagnetic radiation within an absorption spectrum of the fluorescent component and a detector that detects electromagnetic radiation within the tail portion of fluorescence spectrum.

In yet another embodiment, the inventors have recognized that the increased autofluorescence signal of lipopigments relative to healthy tissue (e.g., with little or no lipopigments or corresponding strong autofluorescence) in the NIR/SWIR spectral region may be used to identify a disease state and/or a patient condition. Consequently, in some embodiments, tissue that comprises lipopigments when in a diseased state that does not autofluoresce strongly in these spectral regions may be exposed to an autofluorescence excitation source of the tissue in a first spectral region. The autofluorescence signal of the tissue is then detected and imaged using a detector that is sensitive to electromagnetic radiation in a second different spectral region. In some instances the excitation source emits in the NIR spectral region and the detector is sensitive to electromagnetic radiation in the SWIR spectral region. However, different spectral ranges for both excitation and autofluorescence detection different than those noted above are also contemplated as the disclosure is not so limited.

In certain embodiments, a targeted disease component may exhibit a lower autofluorescence intensity as compared to the area surrounding the targeted disease component. For example, in some cases, the tissue surrounding the targeted disease component (e.g. a tumor) may have a corresponding strong autofluorescence signal due to the presence of a certain disease state (e.g., cirrhosis resulting from hepatotoxicity). Thus, the targeted disease component itself may be detected based on the absence or decreased autofluorescence intensity relative to the surrounding tissue, i.e. negative autofluorescence contrast.

In view of the above, in some embodiments, a device may include an excitation source that is configured and arranged to expose tissue, that may or may not include lipopigments, to electromagnetic radiation within an adsorption spectra of the lipopigments. The excitation source may emit electromagnetic radiation (e.g., excitation light) within a particular band of the absorption spectra of the lipopigments. Alternatively, the excitation source may emit electromagnetic radiation over the entire absorption spectrum of the lipopigments, as the disclosure is not so limited. Regardless, in some embodiments, the excitation source may emit electromagnetic radiation that includes wavelengths longer than or equal to 600 nm, 650 nm, 700 nm, 800 nm, 850 nm, 900 nm or any other appropriate wavelength. Additionally, the excitation source may emit electromagnetic radiation within an absorption spectrum that includes wavelengths shorter than or equal to 1000 nm, 900 nm, 850 nm, 800 nm, 700 nm, and/or any other appropriate wavelength. Combinations of the above range of wavelengths are contemplated, including, for example, an excitation source that emits electromagnetic radiation between or equal to 600 nm and 900 nm, 650 nm and 900 nm, 650 nm and 850 nm, and/or any other appropriate range of wavelengths. Without wishing to be bound by theory, excitation sources that emit radiation in the NIR wavelength range may offer several benefits including, for example, improved tissue penetration depths for the excitation light over visible wavelengths. Accordingly, in some embodiments, an excitation source may emit electromagnetic radiation (e.g., excitation light) with wavelengths between or equal to 650 nm and 900 nm. Further, in some embodiments, when imaging the liver of a subject, it may be desirable to use an excitation source that emits electromagnetic radiation in a range between or equal to 800 nm and 850 nm including, for example, 808 nm, to avoid exciting autofluorescence of other surrounding biological structures such as the gallbladder and gastrointestinal tract. Though a liver may be imaged with other excitation wavelengths as well. While particular ranges and combinations of wavelengths are noted above, it should be understood that other wavelengths for an excitation source, both longer than and shorter than those noted above, are also contemplated as the disclosure is not so limited.

The above noted wavelength ranges may provide increased tissue penetration. However, in some embodiments, such as open surgical procedures as well as endoscopic, catheter, and laparoscopic procedures, it may be acceptable to user shorter wavelengths with less penetration depth. Accordingly, in yet another embodiment, an excitation source may emit electromagnetic radiation with one or more wavelengths between 400 nm and 850 nm. For example, in certain embodiments, the excitation source may emit excitation light with one or more wavelengths longer than or equal to 400 nm, longer than or equal to 450 nm, longer than or equal to 500 nm, or longer than or equal to 550 nm. According to some embodiments, the excitation source may emit excitation light with one or more wavelengths shorter than or equal to 850 nm, shorter than or equal to 600 nm, shorter than or equal to 550 nm, shorter than or equal to 500 nm, or shorter than or equal to 450 nm. Combinations of the above range of wavelengths are also contemplated, such as, for example, an excitation source that emits electromagnetic radiation between or equal to 400 nm and 600 nm or 450 nm and 550 nm.

In view of the forgoing, the Inventors have recognized that in certain embodiments, the systems described herein for the identification and detection of disease states associated with cumulative lipopigments may be implemented in any number of different form factors including, catheters, endoscopes, laparoscopes, fiber-optic instruments, hand held systems for open surgical procedures, overhead mounted imaging systems, table mounted systems, and/or any other appropriate form factor as the disclosure is not so limited.

Based on the identification of the lipopigments as the source of observed autofluorescence, the Inventors have recognized that in some applications, the use of a multi-photon excitation method may be desirable. Specifically, the use of such a method may improve the specificity of the regions excited by the incident excitation light and improved penetration depths due to the use of longer wavelengths. Accordingly, in some embodiments, an excitation source may emit excitation light with wavelengths longer than the imaging wavelengths to which an associated detector is sensitive. For example, in some embodiments, a detector that is sensitive to electromagnetic radiation may be used to image tissue that comprises lipopigments when in a diseased state, and the image is taken of an emission wavelength range (e.g., between 850 and 1200 nm). In some cases, the excitation light emitted by the excitation source may have a range of wavelengths that is longer than the corresponding imaging wavelengths (e.g., 1300 nm, 1400 nm, 1500 nm, etc.). According to certain embodiments, the excitation source emits excitation light with wavelengths between 1000 and 2000 nm as well as any other appropriate range of wavelengths. Correspondingly, a detector may image autofluorescence signals at wavelengths between or equal to 850 nm and 1000 nm, 850 nm, and 900 nm, and/or any other appropriate range of wavelengths.

In one embodiment, a device includes a detector for detecting a portion of a tail portion of an autofluorescence spectrum of lipopigments. While this tail portion may correspond to any range of wavelengths depending on the particular application, in one embodiment, the tail portion includes wavelengths longer than or equal to about 700 nm, 800 nm, 850 nm, 900 nm, 1000 nm, 1100 nm, 1200 nm, 1300 nm, 1400 nm, 1500 nm, 1600 nm, 1700 nm, 1800 nm, 1900 nm and/or any other appropriate wavelength. Correspondingly, the tail portion may include wavelengths that are shorter than or equal to about 2000 nm, 1600 nm, 1500 nm, 1400 nm, 1300 nm, 1200 nm, 1100 nm, 1000 nm, 900 nm, 800 nm and/or any other appropriate wavelength. Combinations of the above noted wavelength ranges of a tail portion of a fluorescence spectrum and the ranges a corresponding detector is sensitive to are contemplated. For example, a detector, as well as any associated filters or other optical components, may be configured and arranged to detect a tail portion of electromagnetic radiation at wavelengths between 700 nm and 2000 nm, 850 nm and 1100 nm, as well as 850 nm and 900 nm. Of course, tail portions of a spectrum, and the detectors used to measure them, that exhibit wavelengths, both shorter and longer than those ranges noted above are contemplated as the disclosure is not so limited. The above noted ranges may also be applied to measuring the autofluorescence signals of healthy and diseased tissues as well.

It should be understood that any detector that is sensitive to the desired ranges of electromagnetic radiation described herein may be used with the disclosed devices and methods. However, in one embodiment, a detector used within an imaging and/or diagnostic device may be an InGaAs (Indium Gallium Arsenide) detector, Germanium detector, MCT (Mercury Cadmium Telluride) detector, bolometers, and/or any other appropriate detector that is sensitive to the range of electromagnetic wavelengths of interest. Additionally, the Inventors have recognized that the identified lipopigments exhibit relatively strong autofluorescence signal intensities at longer wavelengths than previously realized. Accordingly, in some embodiments, more typical detectors capable of operating at shorter wavelengths than the above-noted detectors may be used. For example, in one embodiment, a silicon-based detector capable of detecting electric magnetic radiation at wavelengths between about 600 nm and 900 nm may be used. However, it should be understood that a detector is not limited to only these types of detectors, and in some instances a detector may be a combination of detectors covering a range of wavelengths that may be within the noted wavelength ranges, and/or may extend outside of the described ranges, as the disclosure is not so limited. The detectors described herein may detect low-quantum yield autofluorescence signals at a level comparable to that obtained with high quantum yield exogenous markers. In some cases, detection of low-quantum yield autofluorescence is performed through lower energy NIR excitation, which is less destructive to and interacts less with biological tissues and/or cells.

In the above noted embodiments, a corresponding peak emission wavelength of an autofluorescent spectrum of a lipopigments component may be at shorter wavelengths than a tail portion of the fluorescent component. For instance, in some embodiments, a peak wavelength of a fluorescent component may be at wavelengths shorter than or equal to 900 nm, 800 nm, 700 nm, or any other appropriate wavelength depending on the particular lipopigments of interest.

Fluorescence and autofluorescence spectra typically include one or more peak fluorescence intensities. These intensities may either be a local and/or global peak that is greater than the intensity of the fluorescence spectrum at surrounding wavelengths. Further, while a spectrum is continuous and a peak will span a range of wavelengths, a peak is generally described in reference to the wavelength at which the largest fluorescence intensity is measured. Additionally, while it can be argued that a fluorescence spectrum is continuous over all wavelengths, just with a vanishingly small detectable signal, for purposes of this application, wavelengths where the measured fluorescence and/or autofluorescence intensities are less than or equal to 0.01%, 0.1%, 1%, or other appropriate percentage of a maximum peak intensity and/or that are not greater than a background noise of a measured fluorescence signal are not considered as being within a fluorescent and/or autofluorescent spectrum wavelength range, let alone a tail portion of a spectrum, for a particular fluorescent component and/or tissue.

In addition to the above, in some embodiments, a tail portion of a spectrum may be described as corresponding to a portion of the spectrum removed from a global peak of the spectrum. Also, in some embodiments, a tail portion of a spectrum may be described relative to a percentage of an area of a fluorescent spectrum at wavelengths longer than a global peak. For example, in some embodiments, the tail portion of a fluorescent spectrum may be greater than or equal to 1%, 5%, 10%, 20%, or any other appropriate percentage of the area of a fluorescent spectrum. Correspondingly, depending on the location of the spectrum peak, a tail portion of a fluorescence spectrum may be less than or equal to about 70%, 60%, 50%, 40%, 30%, 20%, or any other appropriate percentage of the area of the fluorescence spectrum. Combinations of the above are contemplated including, for example, a tail portion of a fluorescence and/or autofluorescence spectrum that is between about 1% and 40% of the area of the fluorescence spectrum.

Depending on the desired application of the disclosed methods and devices, it may be desirable to either display, and/or further process, an autofluorescence signal obtained by a detector. Therefore, in one embodiment, a detector may output a detected autofluorescence signal to a computing device for further evaluation as detailed below. In addition, or alternatively to the above, the detector may output the detected autofluorescence signals to an associated display such as a monitor or printer. This display may either be separate from an imaging or diagnostic device or it may be integrated therewith as the disclosure is not so limited.

In embodiments, where an autofluorescence signal is output to a computing device, one or more actions may be taken with the received information. For example, in some embodiments, the computing device may either store the information in associated memory for later processing and/or communicate the information to a server, a separate computing device connected to the system, and/or transmit the information to a remotely located computing device for further processing, storage, and/or other handling of the information. In either case, in some embodiments, it may be desirable to process the received signal to determine a condition of the tissue and/or subject. For example, a field of view (FOV) of the detector may include a plurality of pixels that capture an autofluorescence signal from tissue the detector is oriented toward. The intensity of the detected autofluorescence signal for either each pixel, or groupings of pixels, may be compared to a threshold intensity. Pixels meeting, or exceeding, this threshold intensity may be identified as exhibiting a particular disease state or subject condition. Alternatively, a ratio of autofluorescence intensities of individual pixels may be determined relative to the autofluorescence intensity of surrounding tissue to determine particular disease states.

In the above noted embodiments, a computing device comparing the detected fluorescence and/or autofluorescence intensities of the plurality of pixels to a threshold intensity may assign a tissue state and/or subject condition to one or more pixels meeting, or exceeding, the threshold intensity. This tissue state or condition may then be presented on a display (e.g. an image depicting the tissue states of the imaged tissue), stored within the memory of the computing device, transmitted to another computing device, output as a diagnostic result (i.e. a subject condition is present or not), and/or used in any other appropriate fashion.

While specific types of tissue states and subject conditions are discussed herein, it should be understood that the currently disclosed systems and methods may be applied to any appropriate type of subject condition and/or tissue state. For example, according to certain embodiments, at least a portion of the detected autofluorescence intensity may be compared to an intensity threshold to determine if the tissue is in a diseased state, such as non-alcoholic fatty liver disease, liver cirrhosis, lysosomal storage diseases, or other disease states. In certain embodiments, the intensity threshold may be a baseline autofluorescence intensity threshold based off of clinically determined normal tissue versus diseased tissue autofluorescence intensities. Alternatively, in anther embodiment, the autofluorescence intensity threshold may be based on a prior-obtained autofluorescence intensity of the evaluated tissue. For example, the prior-obtained autofluorescence intensities may be autofluorescence intensity values stored in memory of a particular device that were obtained during a prior imaging session of a particular patient.

In yet another embodiment, the methods described herein may comprise determining a progression state for a patient. As described above, in some cases, at least a portion of the detected autofluorescence signal, i.e. an autofluorescence intensity of at least a portion of the imaged field of view, maybe compared to an intensity threshold to determine if the tissue is in a diseased state. If at least a portion of the detected autofluorescence intensity of the imaged tissue is greater than the intensity threshold, and a diseased state is confirmed, a progression state for a patient may also be determined in some embodiments. In some cases, the progression state for a patient may be determined based on either a difference between the autofluorescence intensity and threshold intensity and/or an area over which the detected autofluorescence signal greater than the threshold intensity is detected. For example, larger areas and/or larger intensities for the detected autofluorescence signals may be associated with different progression states of a detected disease. In one embodiment, progression states may be determined using multiple autofluorescence intensity and/or area thresholds that are associated with the different progression states and the detected autofluorescence signals may be compared to these thresholds to identify a particular progression state. Alternatively, in another embodiment, the autofluorescence signals may be determined as a percentage and/or difference relative to an autofluorescence and/or area threshold associated with a particular disease state. As a non-limiting example, if a detected autofluorescence intensity of an imaged tissue was 10% greater than an intensity and/or area threshold, a progression state associated with this percentage difference may correspond to a lower risk progression state than if the detected autofluorescence signal had an even larger intensity and/or area relative to the threshold intensity.

Methods of determining a progression state for a patient may be useful for monitoring disease states in patients to determine if treatment and/or therapy is necessary. For example, based on a progression state of a patient, the likelihood of disease presence, disease severity, possible treatments and/or medications, as well as follow up frequency (e.g., every three months, six months, etc.) may be determined. Further, due to the relatively noninvasive nature and specificity of the disclosed methods, the methods described herein may decrease the risk of associated errors in disease diagnosis or prognosis (e.g., sample-error), decrease the need for invasive procedures, and improve both pre-clinical testing and in-treatment options for patients.

In view of the above, a progression state of a patient may be used to either provide a determination of a stage or extent of an identified disease state and/or may be used to recommend a course of treatment (e.g., to a medical practitioner). For example, a controller of a device may make the comparison between the detected autofluorescence signals to the stored autofluorescence intensity thresholds and/or area thresholds to determine both a particular disease state and progression state. The controller may then output the identified disease state and/or progression state along with a recommended course of treatment. In one such embodiment, depending on the particular disease state and progression state, recommended courses of treatment may include treatment options, recommending biopsy or other diagnostic procedures, frequency of monitoring (e.g. monitor every 3 months, 6 months, etc.), and/or any other appropriate treatment option. Further, the controller, or other associated computing device, may identify particular autofluorescence parameters associated with a disease state such as the noted intensities, spatial location and/or distribution, areas and/or percentage of coverage, and other appropriate parameters which are associated with a disease state. These parameters may be stored in any appropriate computer readable non-transitory medium in order to monitor these parameters over time to track the progression and/or regression of the disease state over time as well. Such monitoring may be beneficial to a medical practitioner in regards to determining the rate of progression and/or regression of a disease over time, whether or not a patient is responding to treatment, whether treatment is warranted, and/or whether a different treatment should be used.

In addition to the above, it should be noted that while intensity thresholding may be used in some embodiments, in other embodiments, the disclosed systems and methods may be used without intensity thresholding, such as for example, to simply provide an image with better contrasting, as the disclosure is not so limited.

It should be understood that the various devices and methods described herein may be used on any appropriate type of tissue and for any number of different applications. For example, an imaging and/or diagnostic device may be constructed and arranged to both expose tissue within an object to an excitation source of a fluorescent component and/or the tissue itself for subsequently detecting a fluorescence and/or autofluorescence signal from the object. However, depending on the particular use of the device, the object may correspond to a number of different surfaces and/or configurations. In one such embodiment, the object is the body of a subject and the imaging and/or diagnostic device performs noninvasive imaging and/or detection on the subject's entire body at once and/or a subpart of the subject's body (i.e. torso, abdomen, arm, hand, fingers, leg, head, or sub-portions thereof). Alternatively, the object may be a surgical bed of a subject during an operation such as the tissue exposed during an extraction of a cancerous tumor. In yet another embodiment, the object may correspond to an excised piece of tissue (e.g. an excised tumor including tumor margins that are imaged to detect any residual cancer-associated cells). Of course, other specific applications of an imaging and/or diagnostic device are also contemplated as the disclosure is not so limited to only those applications noted herein.

According to certain embodiments, the methods described herein may comprise the application of a quencher that selectively targets lipopigments. In some cases, the quencher may be applied to quench the autofluorescence of the lipopigments (e.g., lipofuscin and/or ceroids) such that the autofluorescence intensity of the substance is decreased in a particular range of wavelengths. Depending on the particular embodiment, the quencher may decrease in intensity the substance in a desired range of wavelengths by at least 40%, 30%, 60%, 70%, 80%, 90%, and/or any other appropriate percentage as the disclosure is not so limited.

A quencher may quench the autofluorescence intensity of lipopigments over any appropriate range of wavelengths including, for example, wavelengths that are shorter than a range of wavelengths associated with a fluorescence signal being imaged by an associated detector. In one such embodiment, the quencher may quench the autofluorescence of the lipopigments in the visible range, but not in the NIR/SWIR range that a detector images. For instance, a quencher may quench the autofluorescence signal intensity of the lipopigments at wavelengths that are shorter than or equal to 800 nm, 750 nm, 700 nm, 600 nm, and or any other appropriate range of wavelengths. Correspondingly, the quencher may quench the autofluorescence signal intensity at wavelengths that are longer than or equal to 375 nm, 400 nm, 500 nm, 600 nm, and/or any appropriate range of wavelengths. Combinations of the above ranges are contemplated including, for example, a quencher that reduces and observed autofluorescence intensity at wavelengths between or equal to about 375 nm and 800 nm, 600 nm and 800 nm, and/or any other appropriate range wavelengths.

It should be understood that any appropriate type of quencher capable of targeting the desired lipopigments may be used. However, in one embodiment, an example of a quencher may include Sudan Black B. Without wishing to be bound by theory, Sudan Black B absorbs electromagnetic radiation up to approximately 800 nm and may be used to quench the autofluorescence from lipofuscin and/or ceroids up to about this wavelength. According to certain embodiments, NIR/SWIR autofluorescence at wavelengths longer than about 800 nm may still be detected after quenching wavelengths up to about 800 nm with a quencher (e.g., Sudan Black B). By quenching autofluorescence from the lipopigments, in some embodiments, other fluorescent probes, such as fluorescent labeled antibodies, may be applied to the tissue that emit in the visible range from about 400 nm up to wavelengths of about 800 nm may subsequently be applied to the tissue to fluorescently label other tissue structures. This may permit the use of fluorescence imaging of these other probes in wavelengths from about 400 nm to 800 nm while still permitting autofluorescence imaging of the lipopigments (e.g., at wavelengths of 800 nm or longer).

As the term is used herein, near infrared (NIR) may refer to electromagnetic wavelengths between about 750 nm and 1000 nm. Additionally, as used herein, the term shortwave infrared (SWIR) may refer to electromagnetic wavelengths between about 1000 nm and 2000 nm.

Turning now to the figures, several non-limiting embodiments are described in further detail. However, it should be understood that the current disclosure is not limited to only those specific embodiments described herein. Instead, the various disclosed components, features, and methods may be arranged in any suitable combination as the disclosure is not so limited.

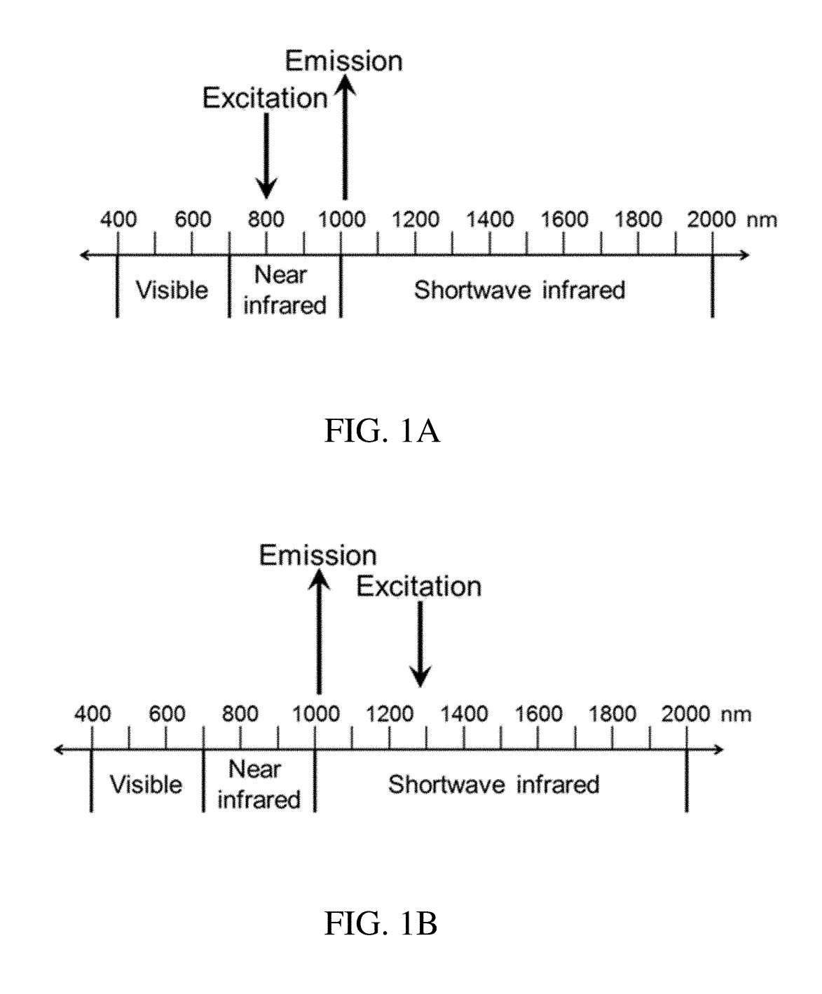

FIG. 1A illustrates a range of different wavelengths corresponding to the different visible, near infrared, and shortwave infrared spectral regions. The figure also illustrates the exposure of tissue to an excitation wavelength, or range of wavelengths, below 850 nm and a corresponding autofluorescent emission that occurs at or above 850 nm. FIG. 1B shows, according to certain embodiments, a graph of an excitation wavelength for a two-photon excitation source and a corresponding emission wavelength. The figure illustrates the exposure of tissue to excitation wavelengths for a two-photon excitation source. The excitation source may emit excitation light with wavelengths longer than the emission wavelength(s) of interest and may be between or equal to 1000 to 2000 nm. In the depicted embodiment, the corresponding autofluorescent emission occurs at 1000 nm. While discrete excitation and emission wavelengths are depicted in the figures, it should be understood that the excitation and emission wavelengths may correspond to a range of wavelengths. Additionally, as described previously, the range of wavelengths may correspond to wavelengths other than those depicted specifically in the figures.

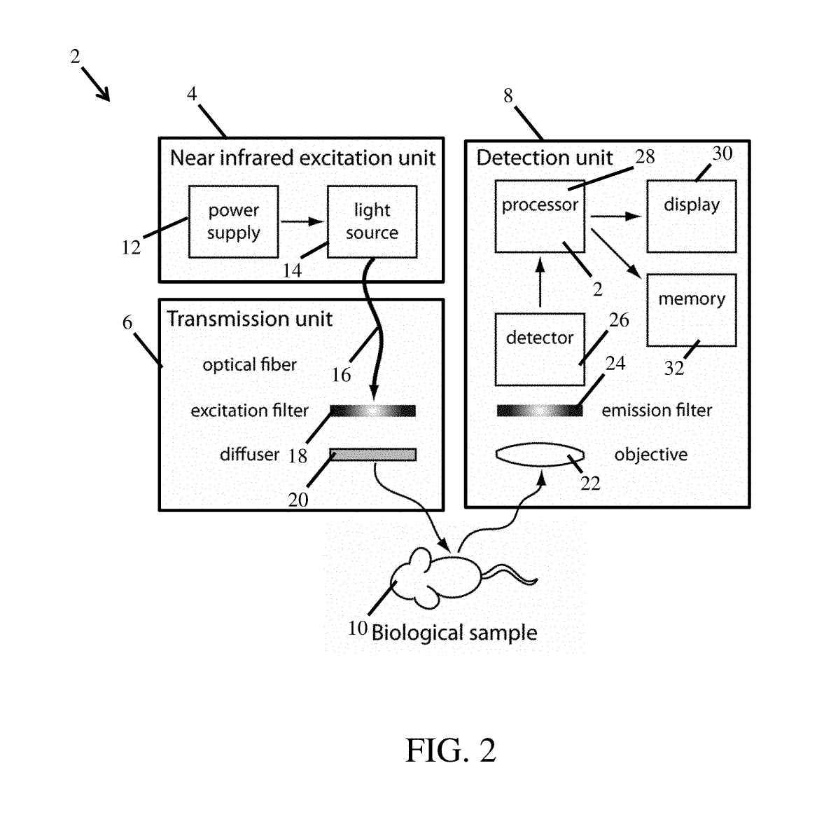

FIG. 2 depicts an imaging and/or diagnostic system 2. The system includes an excitation unit 4, a transmission unit 6, and a detection unit 8. As described further below, the excitation and transmission units expose a portion, or subportion of an object 10, such as the body of a subject, a subportion of the body of a subject, a surgical bed, and/or excised tissue, to an excitation wavelength, or range of wavelengths. Depending on the particular application, the excitation wavelengths may be excitation wavelengths for autofluorescence of the tissue of the object itself. After exposing the object to the excitation wavelengths, the detection unit then detects and processes an autofluorescent signal that is emitted from the object in response to the excitation wavelength. The components and interactions of these units and the object are detailed further below.

In the illustrated embodiment, an excitation unit 4 may include a power source 12 that provides power to an excitation source 14. The excitation source may emit any desired range of wavelengths either within, and sometimes extending out of, an absorption spectrum of an expected lipopigments contained in the tissue of the object 10. Any appropriate type of excitation source may be used including, but not limited to, a laser, light emitting diode, halogen-based emitters, or any other appropriate source of electromagnetic radiation within a desired spectral band. The excitation source is optically coupled to the transmission unit 6 via any appropriate optical coupling 16 including, but not limited to, optical fiber bundles, a light pipe, a planar light guide, an optically clear path, or any other appropriate way of coupling the excitation source to the transmission unit. According to certain embodiments, the optical fiber bundles guide excitation light from an excitation source. Regardless of the specific coupling, in the depicted embodiment, the optical coupling routes the electromagnetic radiation from the excitation source to an excitation filter 18, or set of filters. In certain embodiments, the excitation filer blocks undesirable excitation wavelengths. Depending on the desired excitation wavelengths and the type of source used, the filter may be a combination of low and/or high pass filters to provide electromagnetic radiation within a desired spectrum. For example, the filters may exclude electromagnetic wavelengths above and/or below a desired fluorescence spectrum wavelength or other undesirable excitation wavelengths. In some embodiments, the emitted electromagnetic radiation may then pass through a diffuser to aid in spreading the excitation light across the object.

Once the excitation source has been exposed to the object, components within the tissue itself, such as the noted lipopigments, may autofluorescence emitting electromagnetic radiation within a predetermined fluorescence spectrum towards the appropriately configured and arranged detection unit 8. In the depicted embodiment, the detection unit may include an objective lens 22 to appropriately image the object 10 onto an optically coupled detector 26 that includes a plurality of pixels that image and/or detect an intensity signal from the corresponding portions of the imaged object. Somewhere along the optical path between the object and the detector, one or more filters 24 may be located to exclude reflected excitation light from being detected by the detector. While the detector may be sensitive to any appropriate range of electromagnetic wavelengths, in some embodiments, the detector is sensitive to the ranges of wavelengths described herein including, but not limited to, the shortwave infrared spectral range (e.g., 1000-2000 nm).

Once an autofluorescent signal has been detected by the detector 26, the detector may output the signal to a processor 28. The processor may then appropriately process the information as previously described to determine whether the detected signal corresponds to a particular tissue state and/or subject condition. This information may be determined for each pixel either for a single captured image and/or continuously in real time as might occur during imaging of a surgical procedure. The processed information may then be displayed as an image on a display 30 and/or stored within a memory 32 for subsequent viewing and/or usage.

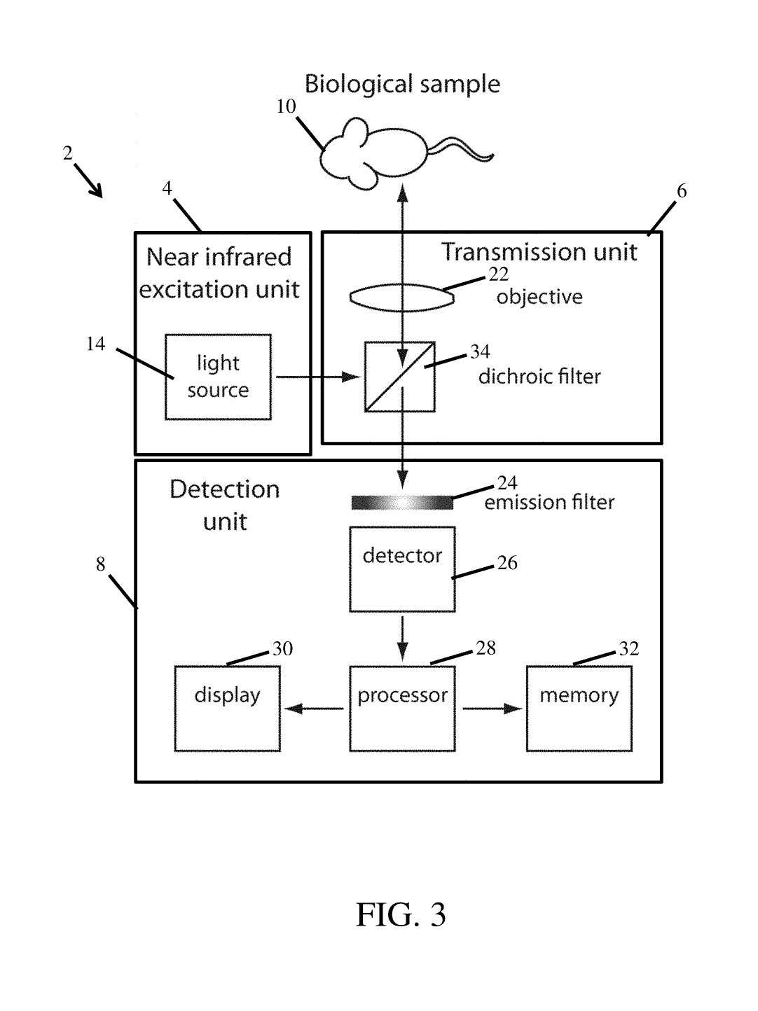

FIG. 3 presents another system similar to that shown in FIG. 2. However, in the depicted embodiment, the device includes a modified transmission unit 6 that includes a dichroic mirror 34 that reflects the desired range of excitation wavelengths towards the object 10 to be imaged. The subsequent autofluorescent signal includes a range of fluorescent wavelengths that pass through an objective lens 22 and are within a pass band of the dichroic mirror such that they pass through the dichroic mirror and are detected by the detector 26 and processed as previously described.

In the above described embodiments, the excitation unit, transmission unit, and detection unit have been described separately. However, it should be understood that these various units may either be combined in a single unitary system, or they may be provided as separate components as the disclosure is not so limited.

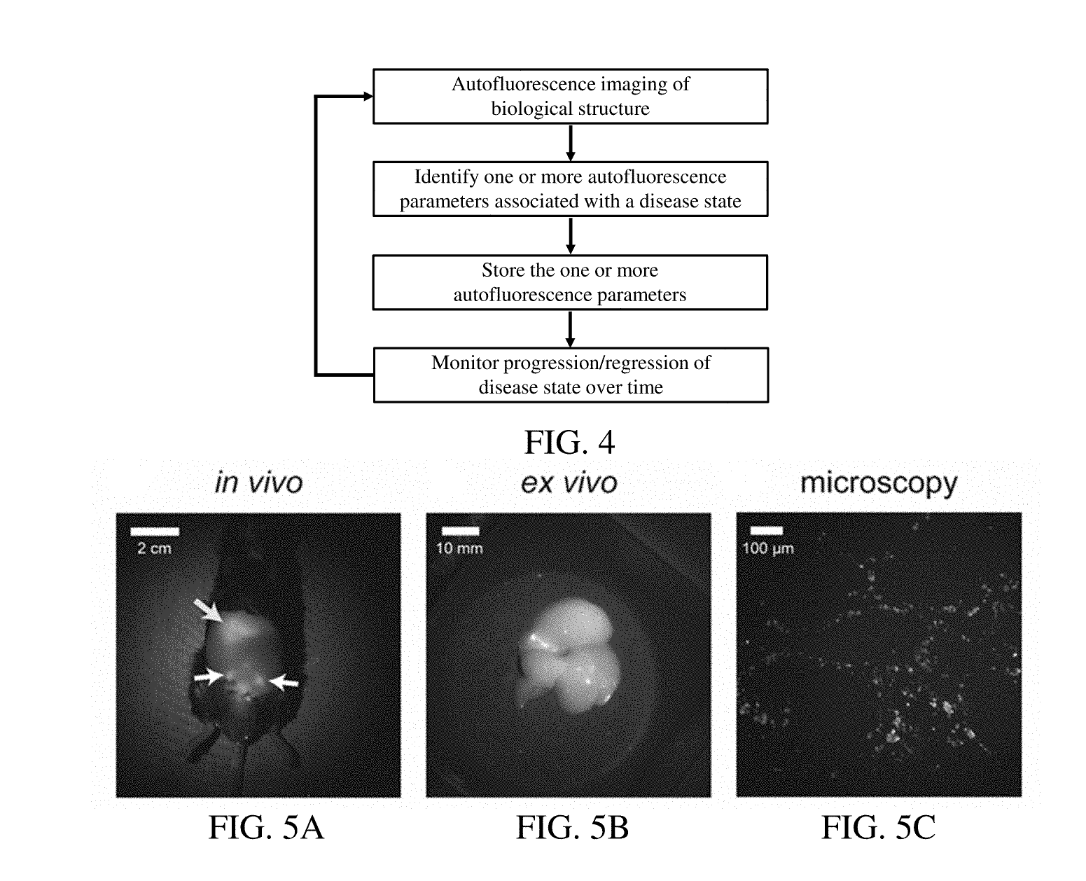

FIG. 4 illustrates one embodiment of a method that may be implemented using the above described systems. In the depicted embodiment, autofluorescence imaging of one or more biological structures may be conducted. During this imaging, one or more autofluorescence parameters associated with a disease state may be identified. This may be done using any appropriate method, metric, or other appropriate standard as the disclosure is not limited in this fashion. For example, a maximum or average signal intensity, percent area of a field of view of an image greater than a threshold signal intensity, anatomical location, spatial distribution of signal greater than a threshold signal intensity, and/or any other appropriate parameter may be identified by a processor of an imaging system. Regardless of the specific metric, the one or more autofluorescence parameters may be stored in a memory of the imaging device, in a memory of a corresponding computing device, and/or in a memory of a remotely located computing device. The one or more autofluorescence parameters may be stored within a data set that includes the corresponding values of the one or more autofluorescence parameters over an appropriate time period. Depending on the particular type of disease state, the time may be on the order of days, weeks, months, and/or years as the disclosure is not limited to any particular time period. In either case, the data set including the values of the one or more autofluorescence parameters over time may be used to monitor the progression and/or regression of a particular disease state over time.

Without wishing to be bound by theory, depending on the particular disease state, the autofluorescence signal may change differently over time with both progression and regression of the disease state. For example, as noted herein, certain disease states may be detected by imaging the autofluorescence associated with lipopigments. However, depending on where and how this pigment is located within the tissue, the autofluorescence of the disease state over time may vary. For example, in disease states where a lipopigment, such as lipofuscin, is mainly located in macrophages which may be easily cleared from the tissue during disease regression, the corresponding autofluorescence signal may decrease over time, which may be correlated with disease regression. However, disease states, in which lipofuscin is present within tissue that is not cleared during disease regression may instead lead to an autofluorescence signal that does not decrease during disease regression, and instead, remains substantially constant during disease regression. In such an embodiment, the autofluorescence signal may be representative of a cumulative stress and/or damage that has occurred to the biological structure being imaged. In either case, autofluorescence imaging may be useful for tracking the regression and/or progression of various disease states depending on where and how a particular lipopigment is deposited within tissue.

In view of the above, in some embodiments, and depending on the particular disease state, changes in autofluorescence parameters such as an increasing area, an increasing maximum or average autofluorescence intensity, and/or similar types of metrics may be indicative of a particular disease state progressing over time. Correspondingly, changes in autofluorescence parameters such as a decreasing area, decreasing maximum or average autofluorescence intensity, and/or other similar types of metrics may be indicative of a particular disease state regressing over time. Accordingly, the data sets corresponding to the stored one or more autofluorescence parameters over time may be output to a medical, or non-medical, practitioner using any appropriate output device, including, for example, a monitor, printer, or other similar type of display method. Based on this output, the practitioner may make more informed decisions regarding prognosis, treatment options, and response to treatments for their patients or subjects based on the methods and systems disclosed herein.

As noted previously, the systems and methods described herein may be implemented in devices that may be used for real-time live cell analysis. Further, in certain embodiments, the devices may be constructed such that they may perform in vivo imaging and detection of various disease states without the need for either open surgery and/or sample excision. For example, the various excitation units, transmission unit, and detection unit may be incorporated into a device that is constructed and arranged to image tissue with appropriate excitation wavelengths and imaging wavelengths to penetrate through sufficient amounts of tissue to image the anatomical structures of interest underlying intervening tissue such as the skin of a subject. For example, wavelengths longer than about 600 nm may be used in certain embodiments to provide increased imaging depths for the disclosed systems. However, it should be understood that embodiments in which the systems and methods described herein are incorporated into a device used during open surgery and/or for imaging of excised samples are also contemplated as the disclosure is not so limited.

In some cases, the above described medical imaging devices may be used to determine if tissue is in one or more disease states. Again, non-limiting examples of possible disease states have been provided above including, but not limited to, non-alcoholic fatty liver disease and/or lysosomal storage diseases. For example, the system may be used to image tissue that comprises lipopigments (e.g., lipofuscin, ceroid, and/or lipofuscin-like lipopigments) when in a diseased state. Furthermore, the system may be configured such that an autofluorescence signal from the imaged tissue may be compared to an intensity threshold to determine if the tissue is in a diseased state. In some embodiments, the device is further configured to determine a progression state for a patient. As described previously, the progression state may be determined based on a magnitude of the autofluorescence intensity and/or an area over which the autofluorescence signal is observed.

The above-described embodiments and examples of the technology described herein can be implemented in any of numerous ways. For example, the embodiments may be implemented using hardware, software or a combination thereof. When implemented in software, the software code can be executed on any suitable processor or collection of processors, whether provided in a single computing device or distributed among multiple computing devices. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semicustom or custom. As a specific example, some commercially available microprocessors have multiple cores such that one or a subset of those cores may constitute a processor. Though, a processor may be implemented using circuitry in any suitable format.

Further, it should be appreciated that a computing device may be embodied in any of a number of forms, such as a rack-mounted computer, a desktop computer, a laptop computer, or a tablet computer. Additionally, a computing device may be embedded in a device not generally regarded as a computing device but with suitable processing capabilities, including a Personal Digital Assistant (PDA), a smart phone or any other suitable portable or fixed electronic device.

Also, a computing device may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Such computing devices may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the disclosed embodiments may be embodied as a computer readable storage medium (or multiple computer readable media) (e.g., a computer memory, one or more floppy discs, compact discs, optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present disclosure as discussed above. As used herein, the term “computer-readable storage medium” encompasses only a non-transitory computer-readable medium that can be considered to be a manufacture (i.e., article of manufacture) or a machine. Alternatively or additionally, the disclosed methods may be embodied as a computer readable medium other than a computer-readable storage medium, such as a propagating signal.

The terms “program” or “software” are used herein in a generic sense to refer to any type of computer code or set of computer-executable instructions that can be employed to program a computing device or other processor to implement various aspects of the present disclosure as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

### Example: NIR/SWIR Autofluorescence

The following example describes detection of NIR/SWIR autofluorescence. The method of detection involved illuminating a region of interest with incident light beams of a NIR excitation source, sensing fluorescence from the tissue with a NIR/SWIR detector, and determining a presence of irregular tissue in a region of interest based on a relatively high fluorescence signal. Specifically, FIG. 5A, shows in vivo NIR/SWIR autofluorescence detected in the liver (top arrow) and in the genitourinary anatomy (bottom arrows) of a mouse model of non-alcoholic fatty liver disease provided with a 12 week choline-deficient, L-amino acid-defined, high-fat diet (CDAHFD). FIG. 5B shows NIR/SWIR autofluorescence detected in the excised liver of the mouse model shown in FIG. 5A. FIG. 5C shows NIR/SWIR autofluorescent cells detected via microscopy in 5 μm slides of formaldehyde-fixed, paraffin-embedded liver tissue from a cirrhosis mouse model provided with 8 weeks of oral CCl₄ and 5% ethanol dosing.

### Example: Detection of Lipopigments Using NIR/SWIR Autofluorescence

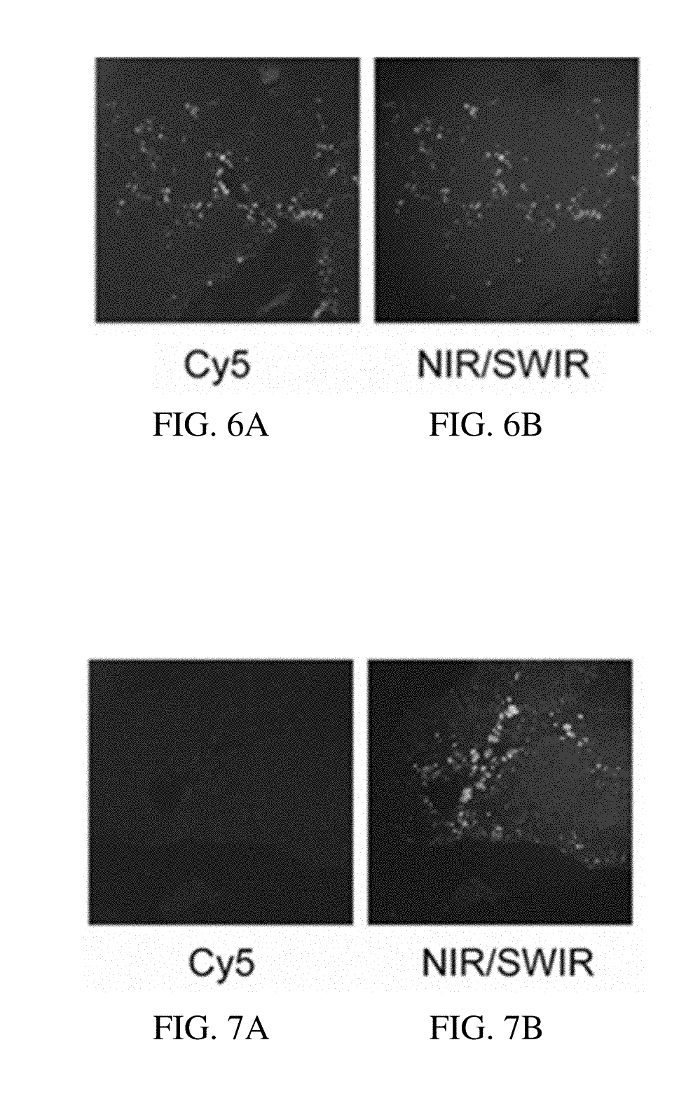

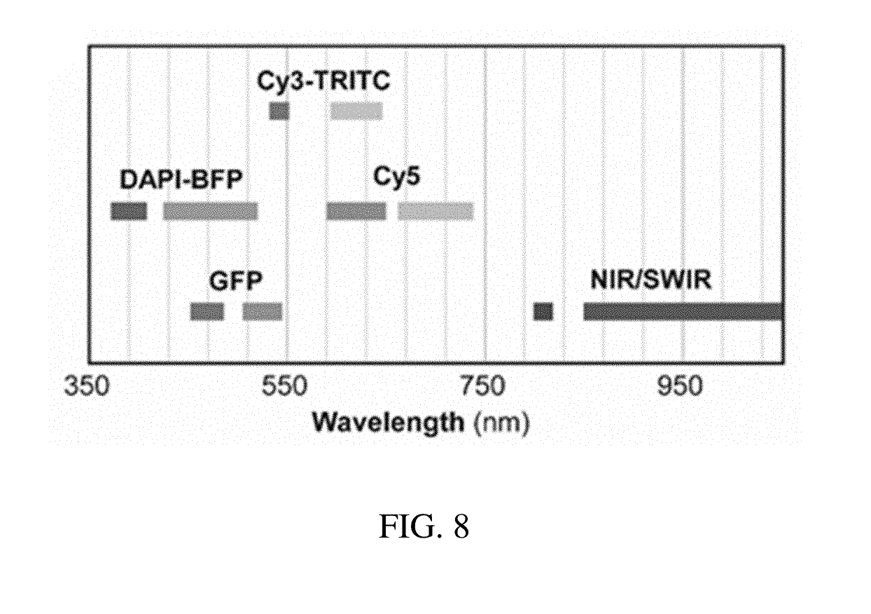

The following example describes the detection of lipopigments by NIR/SWIR autofluorescence in stained and unstained tissue. Most healthy tissue has very little autofluorescence in the NIR and SWIR wavelength ranges. Lipopigments (e.g., lipofuscin and/or ceroid), however, can emit NIR- and SWIR-light, resulting in elevated NIR/SWIR autofluorescence signals under particular disease conditions, providing disease-correlated contrasted images. For example, FIGS. 6A and 6B show spatially-resolved emission detection via microscopy of cirrhotic liver tissue for wavelengths corresponding to the Cy5 channel and NIR/SWIR channel shown in FIG. 8 which shows a diagram of the excitation and emission wavelengths for each microscope filter cube setting. In FIG. 8, for each channel, the excitation wavelengths are represented by the band on the left and the emission wavelengths are represented by the band on the right. Comparatively, FIGS. 7A and 7B show spatially-resolved emission detection via microscopy of formaldehyde-fixed, paraffin-embedded cirrhotic liver tissue with a Sudan Black B stain, which quenched visible wavelength emission of lipofuscin/ceroid through absorption. Autofluorescence was detectable in the Cy5 and NIR/SWIR channels for the unstained sections (as well as Cy3-TRITC channels, not shown here), but was only detectable in the NIR/SWIR channel for stained sections. The same behavior of observable autofluorescence in the unstained tissue and no detectable autofluorescence in the Sudan Black B stained tissue was also observed at wavelengths corresponding to the DAPI-BFP, GFP, and Cy3-TRITC channels. Without wishing to be bound by theory, the autofluorescence signals only being detected in the unquenched samples indicates that it is likely lipofuscin/ceroid that is responsible for the observed autofluorescence due to Sudan Black B targeting and quenching this molecule.

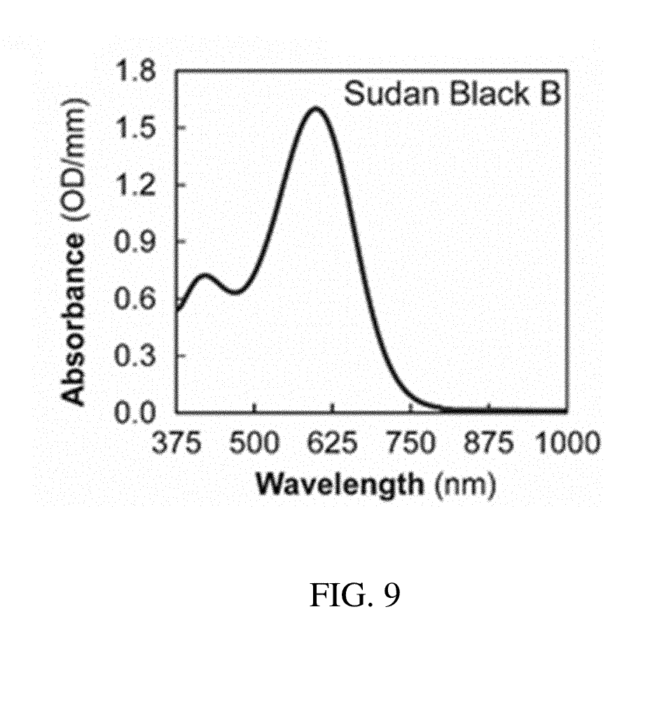

FIG. 9 shows the absorption spectrum of Sudan Black B. As seen in the figure, the quencher absorbs visible light up to approximately 800 nm, which is the mechanism of lipofuscin emission quenching, but transmits light with wavelengths in the NIR/SWIR ranges. Thus Sudan Black B allows the signal to be detected in the NIR/SWIR wavelength ranges even for stained sections, as shown in FIG. 6A-FIG. 7B.

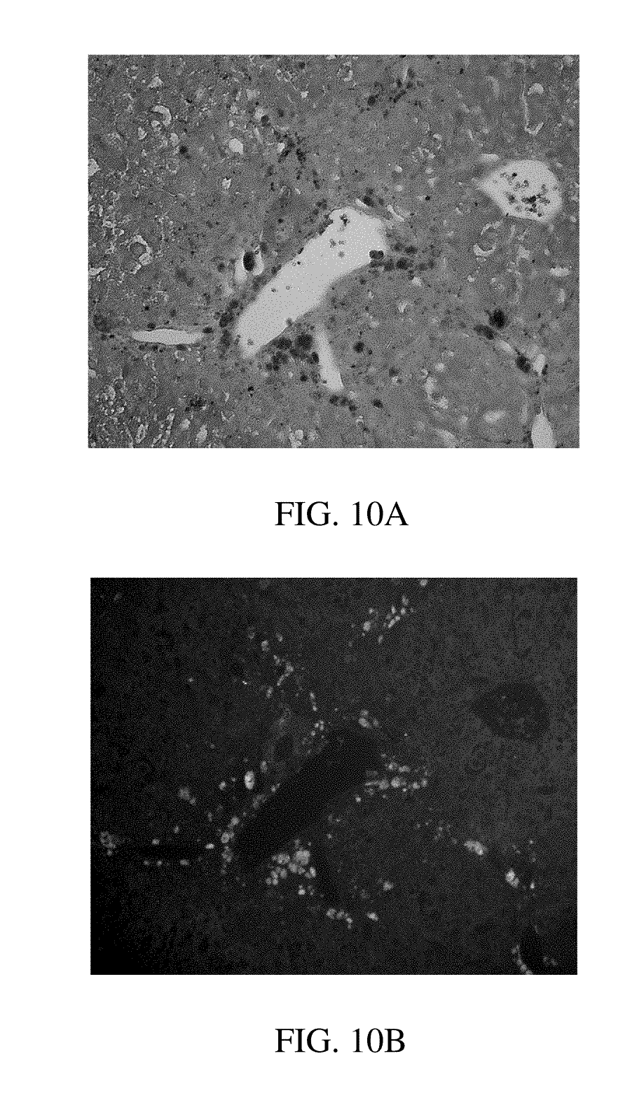

FIG. 10A shows cirrhotic liver tissue (paraformaldehyde-fixed and paraffin embedded) from a cirrhosis mouse model where cirrhosis was induced via an 8 week diet of a CCl₄ and 5% ethanol. The tissue was stained with Sudan Black B. Under visible illumination and detection, areas of lipofuscin in FIG. 10A are indicated by dark-grey/black pigment. The same tissue slice is shown in FIG. 10B, using 808 nm excitation and NIR/SWIR detection showing autofluorescence of the Sudan Black B-positive areas. This illustrates the viability of quenching lipofuscin at visible wavelength ranges while still detecting the autofluorescence signal in the desired NIR/SWIR wavelength ranges.

In the above example, it is noted that Sudan Black B staining is classically performed in non-paraffin embedded tissues, wherein Sudan Black B reveals a totally different staining pattern, since fat/lipids are stained as well. These are lost in the process of paraffin embedding, hence making Sudan Black B specific for lipofuscin which is resilient to these processing steps.

### Example: Detection of Non-Alcoholic Fatty Liver Disease

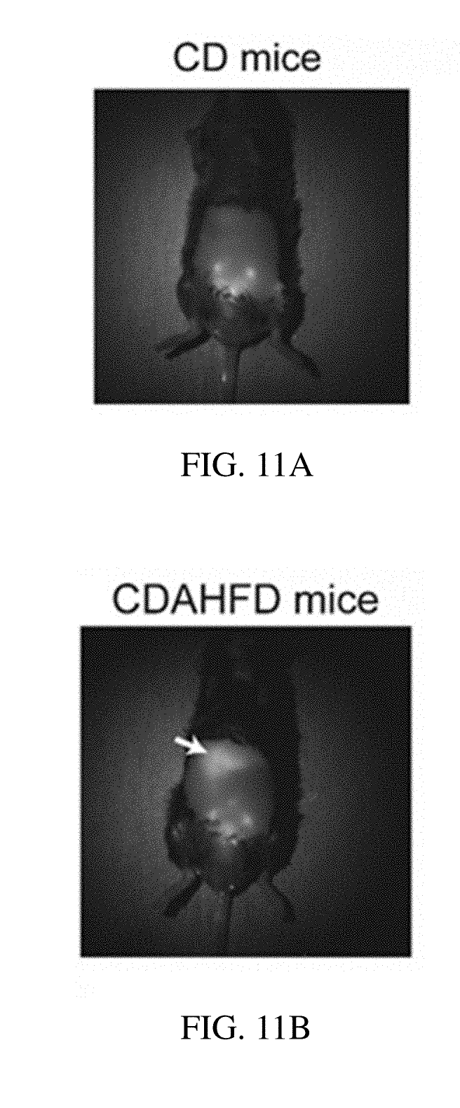

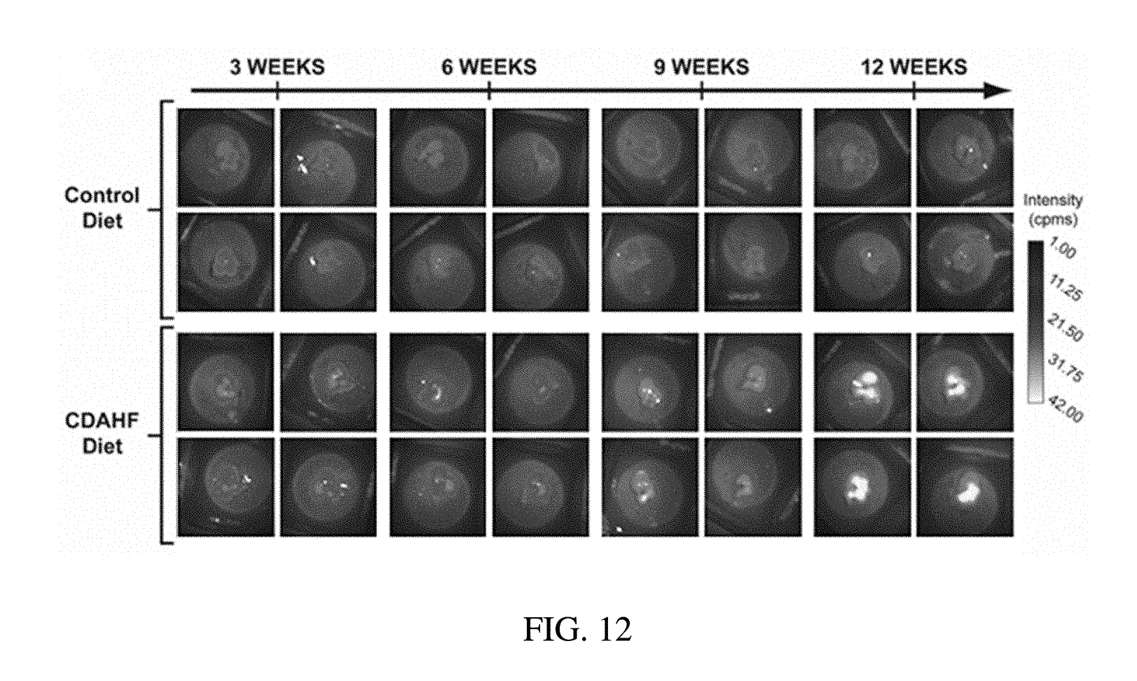

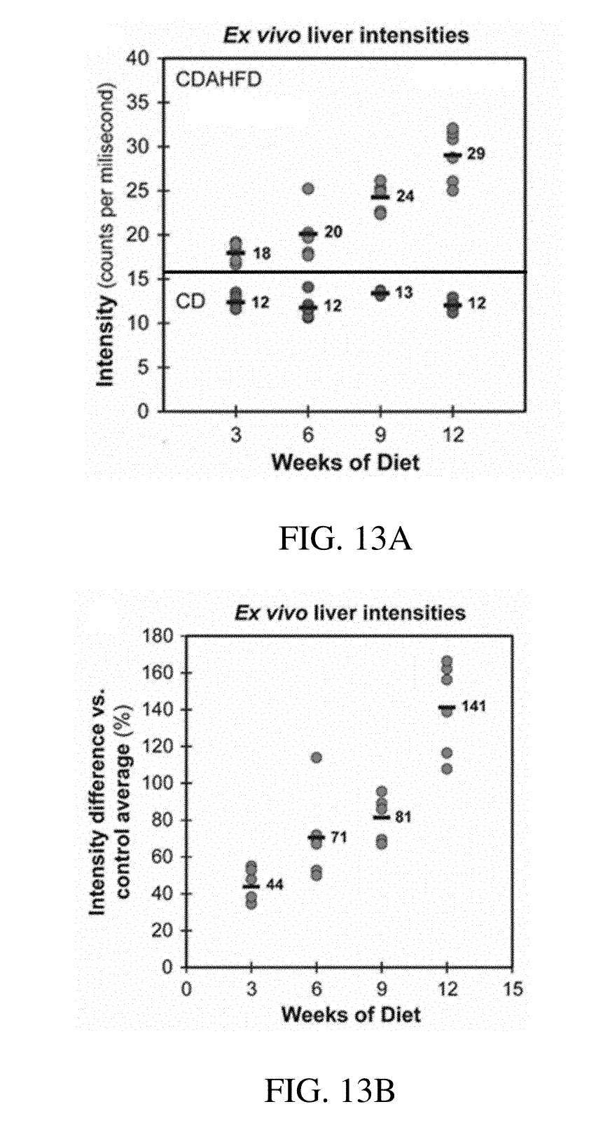

The following example describes the observance of NIR/SWIR autofluorescence signals attributed to lipofuscin/ceroids in diseased animal models. Strong NIR/SWIR autofluorescence signals sufficient for in vivo signal detection were found in both the livers of non-alcoholic fatty liver disease (NAFLD) mouse models and the livers of cirrhosis mouse models. For example, mice were fed 12 weeks of either a control diet (CD), or a choline-deficient, L-amino acid-defined, high-fat diet (CDAHFD) meant to induce non-alcoholic fatty liver disease. As shown in FIGS. 11A and 11B, the mice fed a CD are clearly distinguishable from the mice fed the CDAHFD based on the intensity of signal in the liver, as shown by the arrow. FIGS. 11A and 11B also show the simultaneous presence of NIR/SWIR autofluorescence of the male reproductive tract as well. FIG. 12 shows representative images of the ex vivo livers of mice on their respective diets (CD, top, vs. CDAHFD, bottom) for 3 weeks, 6 weeks, 9 weeks, and 12 weeks. The livers of the mice on the CD (n=24) show on average 12.5 counts per millisecond (cpms) of NIR/SWIR autofluorescence, whereas mice on the CDAHFD show on average approximately 18, 20, 24, and 29 cpms for 3 weeks, 6 weeks, 9 weeks, and 12 weeks of CDAHFD, respectively (n=6 for each time point), as plotted in FIG. 13A. The same quantification of ex vivo liver intensity represented by the percent increase in signal versus the average intensity of the controls at the respective time point is shown in FIG. 13B.

### Example: Real-Time Noninvasive Imaging of CCl₄ Induced Fibrosis

Autofluorescence was investigated in two models of progressive liver fibrosis with distinct disease mechanisms.

The first model was hepatotoxin-induced liver fibrosis where administered carbon tetrachloride (CCl₄) is metabolized into free radicals, which propagate into reactive oxygen species. During experimentation, CCl₄ was administered in olive oil by oral gavage to three cohorts of wild-type C3Hf/Sed mice three times per week for three, six, and eight weeks along with 5% ethanol drinking water ad libitum. Age-matched controls were administered olive oil by oral gavage and supplied with 5% ethanol drinking water.

The second model was based on obstructive cholestosis known to lead to portal fibrosis. Common bile duct ligation (CBDL) surgery was performed to double ligate the common bile duct, causing secondary biliary cirrhosis. Mice were sacrificed four weeks after the procedure, when cirrhosis is known to have developed.

Liver autofluorescence of each disease model and cohort was imaged in vivo through intact, shaved skin using 808 nm excitation, as well as ex vivo in whole excised livers followed by imaging of the paraffin-embedded liver tissue slices.

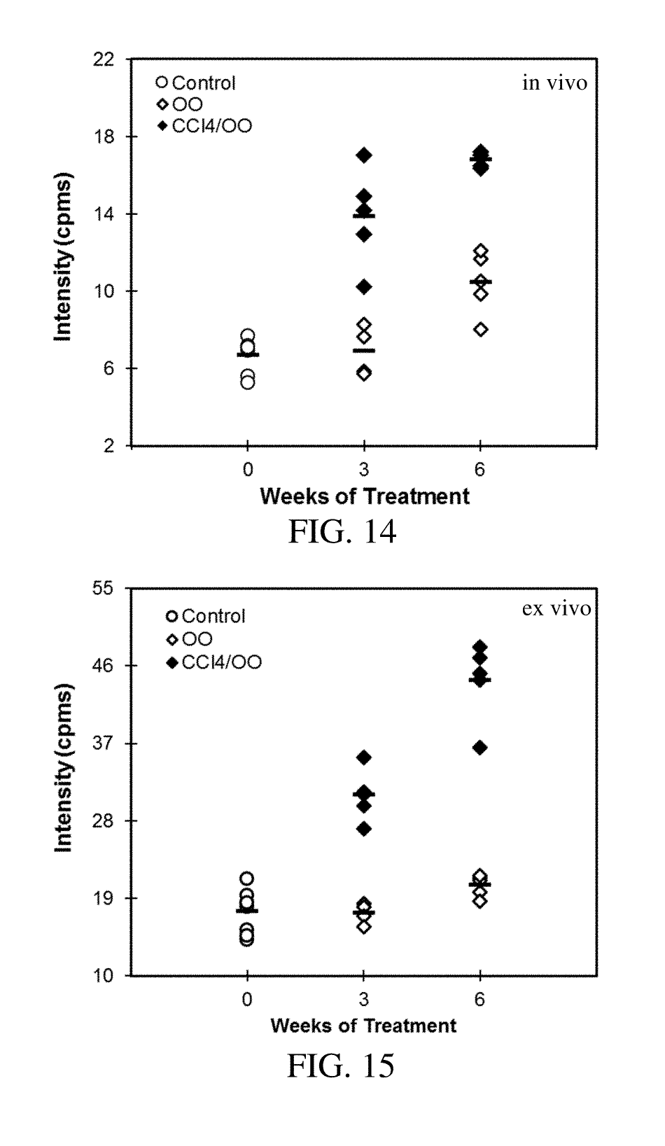

The liver autofluorescence signal of CCl₄-treated mice was clearly distinguishable from age-matched control mice at all measured time-points. Using an InGaAs camera with an 850 nm long pass detection, autofluorescence from the liver was sufficiently strong for real-time imaging at 9.17 frames per second (fps) in awake and freely behaving mice. Further, the in vivo detected liver autofluorescence signal intensity was approximately two-fold higher than in control mice after three weeks of CCl₄ administration (P<0.0016), and six-weeks of CCl₄ administration (P<0.0069), see FIG. 14. Quantification of ex vivo autofluorescence suggests that the liver autofluorescence increases significantly relative to age-matched controls with the ratio between treated and control group signals at 1.8 (P<2.4×10⁻⁴), and 2.2 (P<9.6×10⁻⁵) for 3 week and 6 week groups respectively, see FIG. 15.

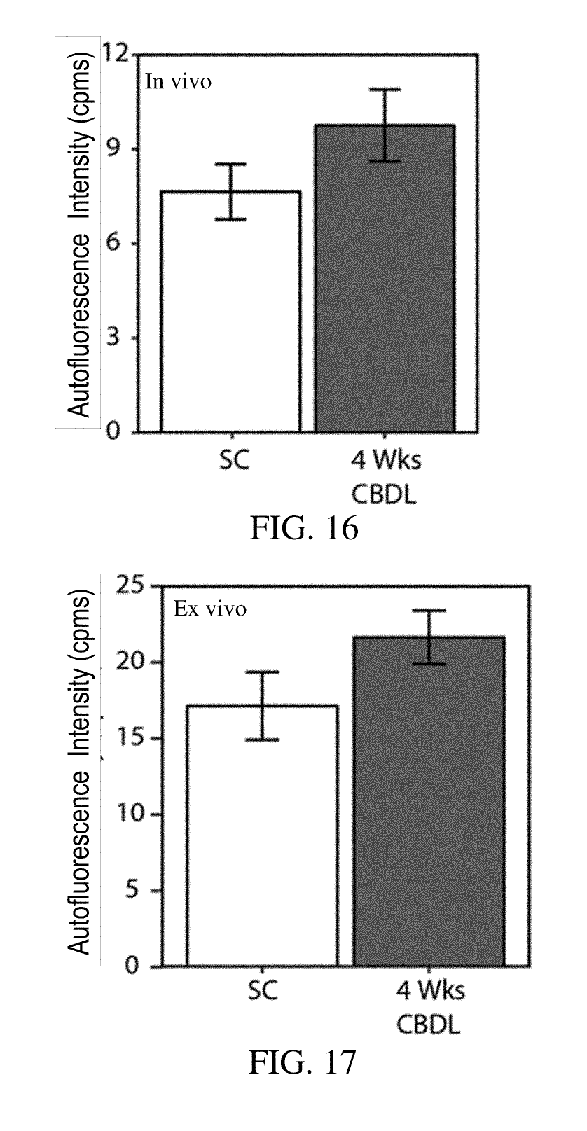

As shown in FIGS. 16 and 17, liver autofluorescence in the CBDL fibrosis model, both in vivo and ex vivo respectively, was on average only slightly elevated compared to age-matched control mice that did not undergo the CBDL procedure by quantification of both in vivo and ex vivo measurements (P_{in vivo}<0.010, P_{ex vivo}<0.018). Furthermore, the absolute liver autofluorescence signal in CBDL mice was substantially less pronounced than that in the CCl₄ mouse model described above. Without wishing to be bound by theory, relative to the CCl₄ model, CBDL mice experience less oxidative stress in the development of fibrosis, and additionally have differences in the main source of myofibroblast production. These results may suggest that the oxidation damage pathway is a factor in the enhancement of autofluorescence with liver disease progression, and point toward an autofluorescent pigment whose formation is linked with oxidation, such as the lipopigment lipofuscin or ceroid. It was further noted that in both CBDL and CCl₄-treated mice, the bile duct and gall bladder were not autofluorescent at the imaged wavelengths, ruling out autofluorescent components of the bile (i.e. biliverdin/bilirubin) as the origin of the disease-associated, NIR/SWIR autofluorescence signal.

The autofluorescence signal was further investigated through microscopy of liver tissue samples from the CCl₄ and CBDL models. It was found that the disease-correlated autofluorescence signal could be detected in unstained, paraffin-embedded liver tissue. This finding further ruled out components of the blood (e.g. porphyrins), which are generally washed out during the paraffin-embedding procedure, as the origin of the signal. All cohorts of the CCl₄-treated mice showed significantly greater percent area of autofluorescence relative to their age-matched controls (˜15-fold increase by six weeks of treatment), in correlation with increasing fibrosis, which was confirmed by Sirius Red staining. Specifically, consistent with both the in vivo and ex vivo imaging, there was a significant difference between mice treated for three weeks and mice treated for six weeks relative to age-matched controls (P_{3 weeks}<3.8×10⁻³, P_{6 weeks}<4.0×10⁻³). Similar to whole liver imaging, CBDL tissue samples, showed a small but significant greater percent area compared with standard chow control mice (P<0.011), again this was substantially less as compared to CCl₄ samples.

### Example: Lipofuscin Autofluorescence

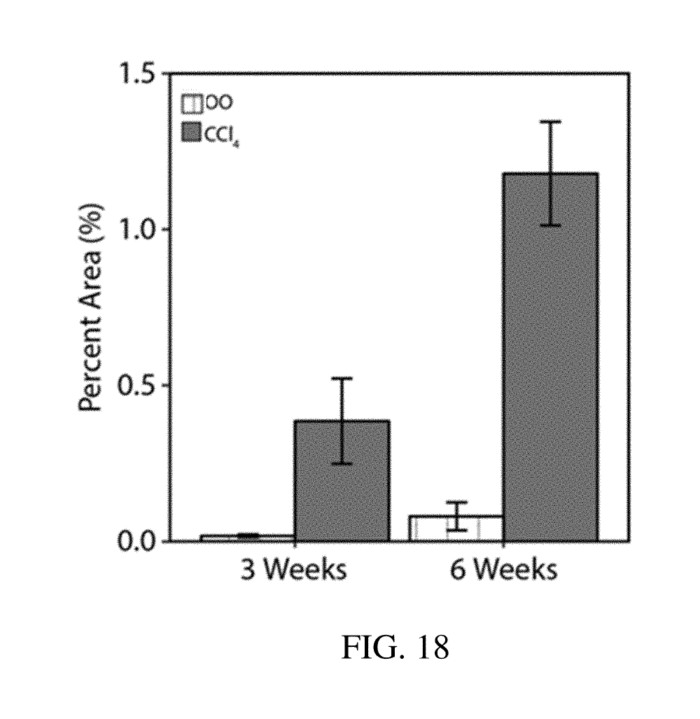

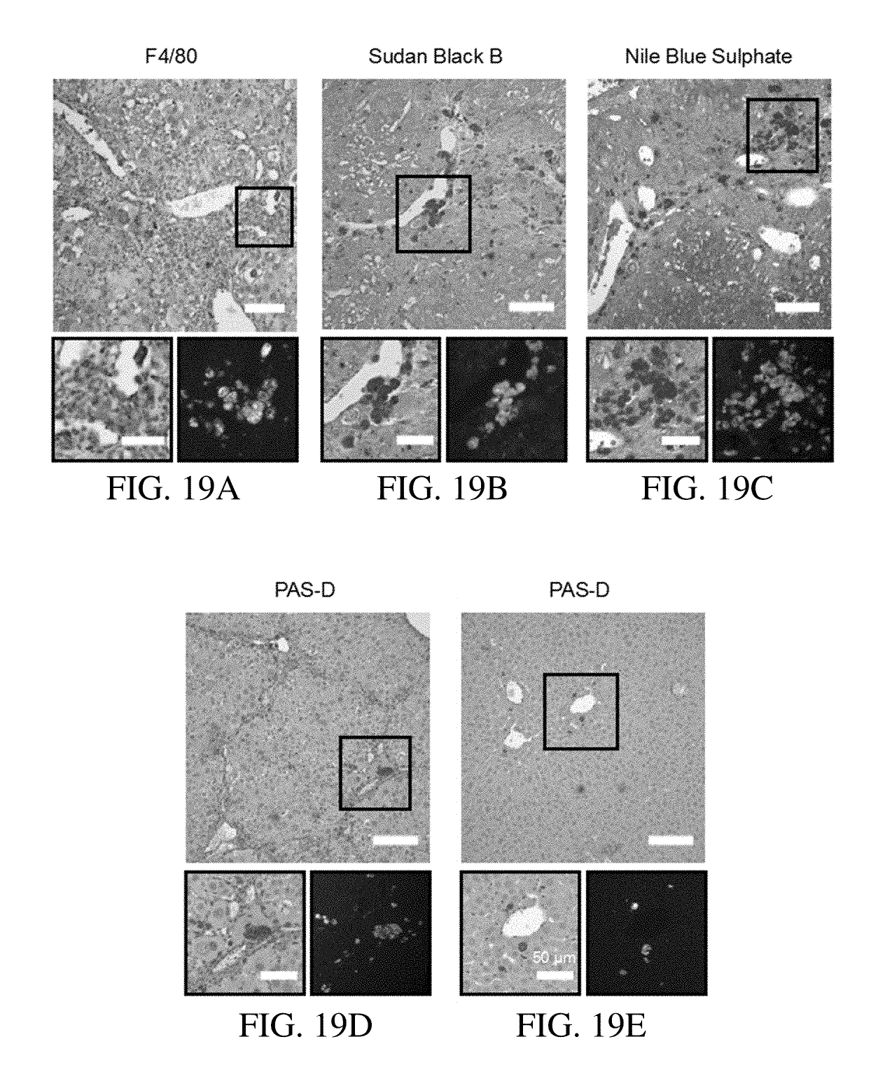

In order to determine the origin of the detected autofluorescence signal, histological staining on ex vivo liver slices from the CCl₄ model was performed. The autofluorescence signal survived deparaffinization and mounting procedures using a variety of mounting media types without substantial quenching, and was detected on deparaffinized, stained, and mounted tissue, indicating a robust signal. FIG. 18 depicts a graph of the percent area of autofluorescence observed in the control versus CCl₄-treated tissue slices. Based on the hematoxylin and eosin (H&E) stains and the pathogenesis of CCl₄-induced cirrhosis, the tissue was stained for the macrophage marker F4/80, the activated hepatic stellate cell (HSC) marker, α-smooth muscle Actin (α-SMA), and the ductular reaction marker CK19. The signal was not correlated with α-SMA or CK19. However, as shown in FIG. 19A, the signal did exhibit a high colocalization with F4/80. The light brown appearance of the cell cytoplasm observed in the images was suggestive of ceroid macrophages, which was even more pronounced in the α-SMA-F4/80 double stain confirming co-localization with F4/80 cell marker, but not with α-SMA positive cells. Ceroid macrophages are known to be associated with liver injury and liver fibrosis. Thus, the autofluorescence response of the tissue was investigated for three known stains for lipofuscin/ceroid lipopigments: Sudan Black B (SBB), Nile Blue A Sulphate (NBS), and periodic acid Schiff reaction after diastase (PAS-D), see FIGS. 19B-19E. The NIR/SWIR autofluorescence was strongly correlated with SBB-positive, NBS-positive, and PAS-D positive regions of the tissue as shown in the figures. In addition, the autofluorescence signal was further tested by de-staining NBS-stained tissue using acetone, and subsequently re-staining with SBB with the autofluorescence signal in the restained tissue overlaying the location of the conventional lipofuscin stain. Thus, without wishing to be bound by theory, the observed oxidative stress-correlated autofluorescence is most likely attributable to lipofuscin/ceroid.

### Example: Noninvasive Tracking of CCl₄ Hepatic Injury Regression

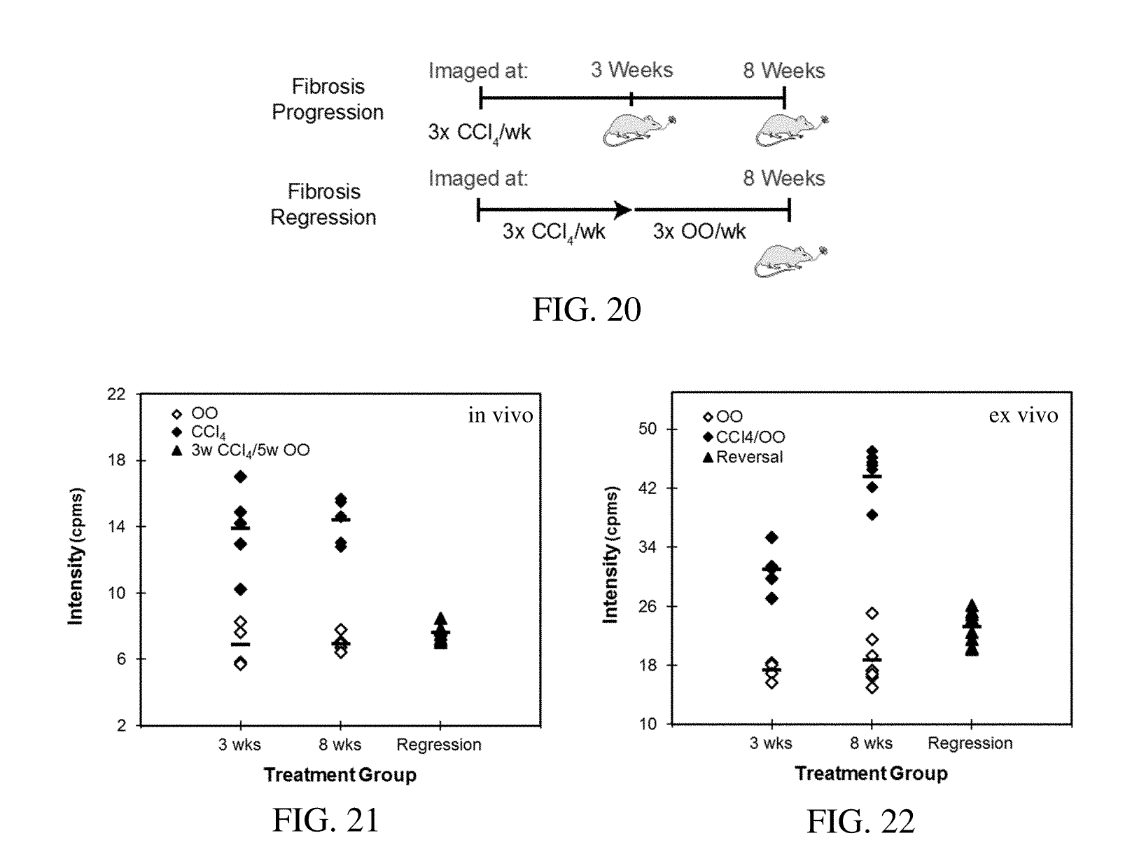

The ability to measure a signal that correlates with disease progression has practical implications for diagnosis and disease staging. Similarly, it may be desirable to have a method of measuring disease regression. In vivo studies tracking the reversibility of activated hepatic stellate cells (HSCs) and macrophages after discontinuing CCl₄-treatment suggest that the correlated autofluorescence could likewise regress. These studies show that activated HSCs are both cleared by apoptosis in addition to reverting to an inactive state, while macrophages play a role in the resolution of both cellular and matrix components of the fibrotic response. Accordingly, the behavior of the autofluorescence signal in response to fibrosis regression by discontinuing CCl₄ treatment and allowing for recovery from CCl₄ hepatic injury was investigated. Specifically, as shown in FIG. 20, CCl₄ was administered in olive oil by oral gavage three times per week for three weeks, followed by five weeks of recovery, during which time only olive oil was administered. This cohort was compared with mice that received CCl₄ treatment for a total of three weeks (presented above), or eight weeks, along with age-matched controls which received only olive oil as also shown in the figure. All mice additionally received 5% ethanol drinking water ad libitum for the duration of the study.

It was found that the autofluorescence intensity was significantly lower in mice that had undergone regression of liver fibrosis in comparison to three weeks or eight-weeks (P_{in vivo}<7.3×10⁻⁶, P_{ex vivo}<2.0×10⁻¹⁰) of continuous CCl₄ treatment, demonstrating reversal of the autofluorescence signal with disease regression both in vivo and ex vivo, see FIGS. 21 and 22 respectively. In vivo autofluorescence of the regression cohort was similar to that of the mice receiving only olive oil which may likely be due to the attenuation and scattering of relatively low levels of autofluorescence by overlying tissue as shown in FIG. 21. However, mice undergoing fibrosis regression or hepatic injury by CCl₄ had significantly higher ex vivo autofluorescence compared to control groups, as shown in FIG. 22.

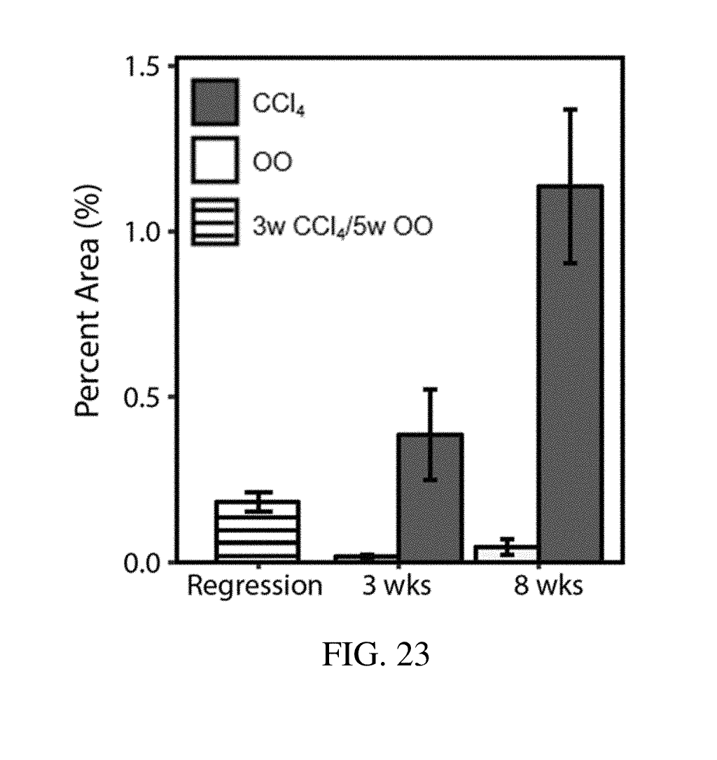

Autofluorescence microscopy of unstained, paraffin-embedded liver tissue slices for the regression model and eight weeks of CCl₄ treatment showed a similar trend as the in vivo and ex vivo measurements. The autofluorescence signal of the regression cohort was lower than mice receiving only three weeks or eight weeks of CCl₄ treatment, but did not fully decrease to the level of non-fibrotic controls, see FIG. 23. Regression of the liver fibrosis after cessation of CCl₄ was substantiated by Sirius Red staining, which indicated a significant reduction of collagen relative to mice with continued CCl₄ treatment. Staining for lipofuscin utilizing PAS/D and SBB, likewise paralleled the observations seen in NIR/SWIR autofluorescence images. H&E stains also showed diminishing number of ceroid macrophages, which are the source of the lipofuscin-induced autofluorescence, in the regression model relative to three weeks of CCl₄ treatment. F4/80 staining further showed a decrease of fibrosis-associated macrophages, as can be expected from literature. Taken together, these results suggest that NIR/SWIR imaging of lipofuscin autofluorescence can provide a quantitative modality to track both the progression and regression of CCl₄-induced hepatic injury.

### Example: Detection of Early Stage NAFLD Via Lipofuscin Autofluorescence

The striking difference in autofluorescence between CCl₄- and CBDL-induced fibrosis may be related to their respective etiopathophysiologies, which in the case of CCl₄, highly depends on oxidative stress, the main driver of lipofuscin formation. Without wishing to be bound by theory, oxidative stress likewise plays an important pathophysiological role in non-alcoholic fatty liver disease (NAFLD), which is one of the leading causes of chronic liver disease (CLD). The disclosed imaging techniques were accordingly used to study a mouse model of NAFLD.

A diet-based NAFLD model was studied in which mice were fed either a control diet (CD) or a choline-deficient, L-amino acid-defined, high-fat diet (CDAHFD) for three, six, nine, twelve, or fifteen weeks (n=6-8 mice per group). After nine to twelve weeks of diet, CDAHFD induces early non-alcoholic steatohepatitis (NASH) with accumulation of fibrosis. The autofluorescence of the liver was imaged in vivo at the end of each diet period, directly followed by excision of the liver tissue for ex vivo imaging, and subsequently by tissue processing.

It was found that NIR/SWIR autofluorescence was detectable at higher levels for both in vivo and ex vivo imaging in the CDAHFD mice compared with CD mice. After nine weeks of diet, the in vivo and ex vivo autofluorescence average signal intensity was 1.2 times and 1.6 times higher, respectively, than age-matched controls (P_{in vivo}<0.017, P_{ex vivo}<3.8×10⁻⁵). By fifteen weeks of diet, the in vivo and ex vivo average signal intensities increased to 1.8 times and 2.8 times higher than controls, respectively (P_{in vivo}<2.2×10⁻⁴, P_{ex vivo}<5.1×10⁻⁶). Quantification of the signal at each imaging time point showed a steady increase of autofluorescence with time, and with progression of disease as indicated by H&E stains. Immunochemically stained CDAHFD liver showed good correlation between PAS/D, F4/80, and SBB stains with the autofluorescent pigment, similar to the findings in the CCl₄ fibrosis model. Furthermore, histological review of liver sections from each diet and control cohort confirmed that CD mice had pathologically normal livers, while CDAHFD mice had severe steatosis from three weeks onwards, with the beginning signs of steatohepatitis and fibrosis (based on H&E and Sirius Red stains) beginning at 12 weeks of diet. As expected, the overall quantity of the autofluorescence signal, as well as the occurrence of the respective stains, were lower than the CCl₄ model.

Analogous to the regression experiments with the CCl₄ fibrosis model, a regression cohort of NAFLD model mice were studied. Specifically, after nine weeks of CDAHFD diet was switched to CD for 6 additional weeks, after which time normalization of the steatosis was expected to occur. Histologically this was evidenced in the experiment by regression of the steatosis and the absence of ballooning of inflammation in the livers of these mice. However, this was not followed by a parallel attenuation of the NIR/SWIR signal at the in vivo and ex vivo levels. Without wishing to be bound by theory, this may be explained by the hepatocellular localization of lipofuscin in NAFLD, which unlike ceroid macrophages in the CCl₄ model, do not rapidly disappear once the injury-inducing treatment is stopped. Hence, the measured signal may reflect the cumulative amount of (oxidative) stress experienced by the liver parenchyma in this parenchymal liver disease.

### Example: Progressive NAFLD Manifestation in IR/IGFR Knockout Lipodystrophy Model

Non-alcoholic fatty liver disease (NAFLD) is also a manifestation of human and rodent models of lipodystrophy. In humans with generalized lipodystrophy, NAFLD often progresses to NASH and cirrhosis, at times requiring liver transplant. The rodent model of lipodystrophy observed in a fat-specific knockout (KO) of the insulin receptor (IR) and insulin-like growth factor 1 receptor (IGFR) is similar to human generalized lipodystrophy, including low leptin levels, insulin resistance, hyperlipidemia, and fatty liver disease. KO mice develop diabetes, hyperlipidemia, and progressive fatty liver disease with hepatomegaly (liver weight up to 25% of body weight), and by 52 weeks of age, show histological evidence of balloon degeneration in the liver in addition to inflammation, fibrosis, and highly dysplastic liver nodules. Thus, autofluorescence in the IR/IGFR-KO mouse model was investigated which may be able to describe the full spectrum of progressive NAFLD and reflect the pathology of NAFLD in humans.

H&E stained liver tissue of wild-type and IR/IGFR-KO mice at 52-weeks of age under 808 nm excitation was imaged, and found that the KO mice exhibited high levels of autofluorescence in the NIR/SWIR region with respect to age-matched wild type mice. The development of severe fibrosis was also accessed by Sirius Red staining. The autofluorescence signal was quantified by percent area in paraffin-embedded samples of wild type (n=4) and KO mice liver tissue (n=5), similar to the method used for CCl₄ and CDAHFD experiments. A 12-fold increase in percent area of the autofluorescence signal in the KO mice (P<0.0009) was found as well as the percent area of Sirius Red. The accumulation of lipofuscin/lipopigments was confirmed using the same set of stains used in the previous two mouse models described above, showing detailed overlap of stained markers and the observed autofluorescence signal.

Hepatocyte balloon degeneration in the IR/IGFR-KO mice is indicative of progressive NAFLD, and by 12-weeks of age these mice have increased levels of reactive oxygen species and lipid peroxidation, which could account for the increased autofluorescence signal when aged to 52-weeks. Furthermore, this model has been shown to express mRNA markers for inflammation and fibrosis by 52 weeks, whereby severe interstitial fibrosis (stage 3 of 4) is present. Thus, the lipodystrophy model supports that increasing autofluorescence with the progression of NAFLD arises from lipopigment molecules, which accumulate due to hepatocyte injury as NAFLD progresses. Similar to the CDAHFD experiments, the accumulation of lipofuscin has punctuate structure and is localized in the hepatocytes, contrasting the fibrosis-associated ceroid macrophages in the CCl₄ model. Without wishing to be bound by theory, the results of this model, in addition to the lipopigment staining, help substantiate that indeed the enhanced signal in the diet-induced NAFLD arises as a consequence of the disease progression.

### Example: Lipofuscin Quantification as a Method to Detect Human Clinical NAFL, NASH, and Cirrhosis

NIR/SWIR autofluorescence imaging was conducted on human tissue samples to assess the potential specificity of the disclosed methods. Specifically, biopsied liver samples were graded based on the degree of steatosis, ballooning, intra-acinar and portal inflammation and fibrosis (including the degree of architectural restructuring). Images were taken of unstained biopsied liver tissue obtained from 15-patients, with ranging histories and stages of NAFL, NASH, and clinically-diagnosed compensated cirrhosis. Autofluorescence in the tissue samples was quantified by determining the percent area of autofluorescence signal using the same thresholding methods as in the mouse experiments. The samples were graded and staged, then separated into NASH and stages of cirrhosis.

While some of the signal resides within the macrophages (Kupffer cells), the majority of the signal arose from punctuate structures in hepatocytes. The distribution and architecture of the autofluorescence signal appeared to follow that of the relatively normal patient tissue, in which lipofuscin accumulates as part of normal age-related processes. Normal tissue at young age (2-3 years old, n=2), exhibited no autofluorescence signal at NIR/SWIR wavelengths, but as early as 20 years of age, the accumulation of lipofuscin in the liver was quantifiable. In diseased liver tissue differences can be appreciated and related to disease stage. For example, in patients diagnosed with cirrhotic liver, the overall lipofuscin signal was significantly decreased compared to normal liver tissue and NAFLD (Stage 1 fibrosis). This is attributed to restructuring of both the architecture of the liver parenchyma in addition to the accumulation of severe fibrosis with thick septa (as compared to the mouse models). Staining for lipofuscin followed the results of the mouse models, with CD68 indicating macrophage distribution and frequency, and PAS/D and SBB showing correlation with lipofuscin and lipopigments.

### Example: Autofluorescence Based Detection of Mucolipidosis Type IV

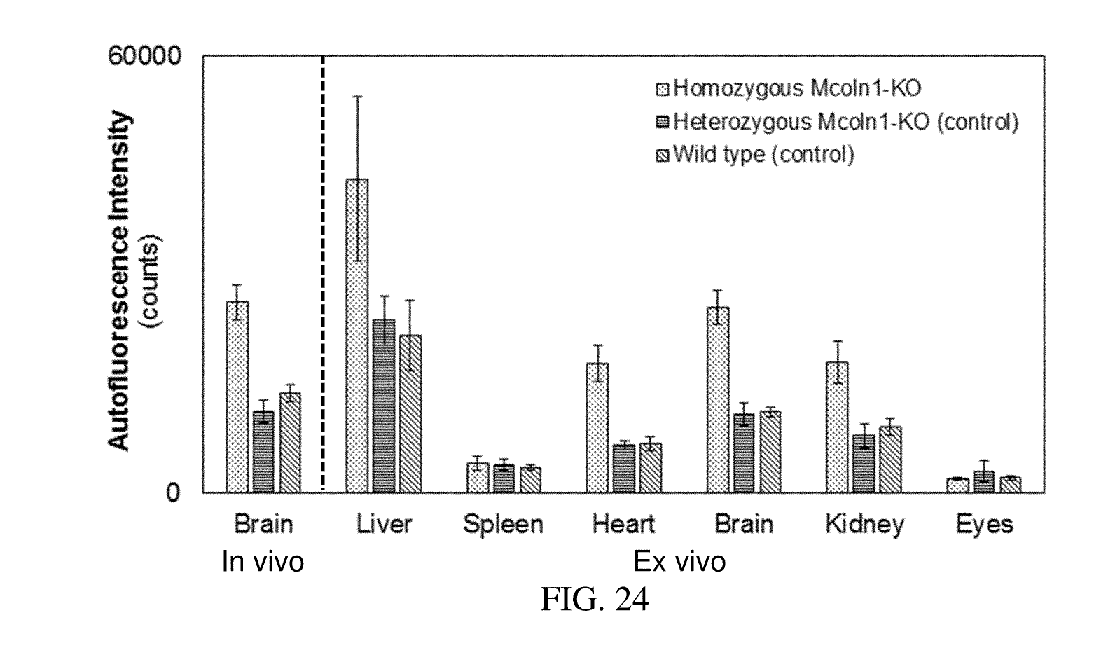

Autofluorescence imaging was used to evaluate homozygous Mcoln1 knock-out mice with mucolipidosis type IV which is a subtype of lysosomal storage disorders. Autofluorescence imaging of pathologically normal wild type and heterozygous Mcoln1 knock-out mice was also conducted for control purposes. Autofluorescence imaging was performed of the brain through an intact skull with the skin removed for each group. After in vivo autofluorescence imaging of the brain, ex vivo autofluorescence imaging of the liver, spleen, heart, brain, kidney, and eyes was subsequently conducted. As shown in FIG. 24, an elevated autofluorescence signal for the homozygous Mcoln1 knock-out cohort was observed both in vivo in the brain, and ex vivo in structures expected to exhibit increased lipofuscin accumulation as compared to the control groups. Specifically, the liver, heart, brain, and kidneys exhibited elevated autofluorescence signals, confirming the ability of the disclosed methods to detect lipofuscin accumulation as a result of a lysosomal storage disorder.

### Example: Lysosomal Storage Diseases

NIR/SWIR autofluorescence was also detected in the brains of 12/15-lipoxygenase knockout mice with consequential upregulated macro-autophagy, a major intracellular degradation pathway that may be linked to an increasing number of (neurodegenerative) diseases such as Huntington's, Parkinson's, and Alzheimer's disease. Based on these results, it is likely that it may be possible to also detect accumulation of lipofuscin-like pigments associated with lysosomal storage diseases, which are inborn errors of metabolism that result in the absence, deficiency, or malfunctioning of an enzyme, leading to the inappropriate storage of material in various cells of the body, as in the neuronal ceroid lipofuscinosis (NCL) family of neurodegenerative disorders or the subgroup of mucolipidosis diseases.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A method, comprising:
exposing tissue that comprises lipopigments when in a diseased state to an excitation source, wherein the lipopigments have at least a portion of an autofluorescence spectrum at wavelengths between 700 nm and 1200 nm; and
imaging the tissue at one or more wavelengths between 700 nm and 1200 nm.

2-4. (canceled)

5. The method of claim 1, further comprising applying a quencher to the lipopigments.

6. The method of claim 5, wherein the quencher reduces an autofluorescence intensity of the lipopigments at wavelengths shorter than the imaged autofluorescence spectrum wavelengths.

7. The method of claim 5, wherein the quencher reduces the autofluorescence intensity of the lipopigments at wavelengths between 400 nm and 800 nm.

8. (canceled)

9. The method of claim 1, wherein the excitation source emits excitation light at one or more wavelengths longer than the imaged autofluorescence wavelengths.

10. (canceled)

11. The method of claim 1, wherein the lipopigments are lipofuscin, ceroid, and/or lipofuscin-like lipopigments.

12. The method of claim 1, further comprising comparing at least a portion of the detected autofluorescence intensity to an intensity threshold to determine if the tissue is in the diseased state.

13-14. (canceled)

15. The method of claim 12, further comprising determining a progression state for a patient.

16. The method of claim 1, wherein the diseased state is a diseased state of non-alcoholic fatty liver disease and/or lysosomal storage diseases.

17. The method of claim 1, further comprising outputting a signal related to the imaged tissue to a display and/or a computing device.

18. (canceled)

19. The method of claim 1, further comprising using a silicon detector to image the tissue.

20. The method of claim 1, further comprising using an Indium Gallium Arsenide detector, a Germanium detector, or a Mercury Cadmium Telluride detector to image the tissue.

21. The method of claim 1, further comprising identifying one or more autofluorescence parameters using the imaged tissue, and storing the one or more autofluorescence parameters in a non-transitory computer-readable medium to monitor the progression and/or regression of the disease state over time.

22. A method, comprising:
exposing tissue that comprises lipopigments when in a diseased state to an excitation source, wherein the lipopigments have at least a portion of an autofluorescence spectrum at wavelengths between 700 nm and 2000 nm, and wherein the diseased state is at least one of non-alcoholic fatty liver disease and/or lysosomal storage diseases;
imaging the tissue at one or more wavelengths between 700 nm and 2000 nm; and
comparing the imaged tissue to an intensity threshold to determine if the tissue is in the diseased state of non-alcoholic fatty liver disease and/or lysosomal storage diseases.

23-26. (canceled)

27. The method of claim 22, further comprising applying a quencher to the lipopigments.

28. The method of claim 27, wherein the quencher reduces an autofluorescence intensity of the lipopigments at wavelengths shorter than the imaged autofluorescence spectrum wavelengths.

29. The method of claim 27, wherein the quencher reduces the autofluorescence intensity of the lipopigments at wavelengths between 400 and 800 nm.

30-32. (canceled)

33. The method of claim 22, wherein the excitation source emits excitation light at one or more wavelengths longer than the imaged autofluorescence wavelengths.

34. (canceled)

35. The method of claim 22, wherein the lipopigments are lipofuscin, ceroid, and/or lipofuscin-like lipopigments.

36. The method of claim 22, further comprising outputting a signal related to the imaged tissue to a display and/or a computing device.

37. (canceled)

38. The method of claim 22, further comprising using as silicon detector to image the tissue.

39. The method of claim 22, further comprising using a InGaAs detector, Germanium detector, or a MCT detector to image the tissue.

40. The method of claim 22, further comprising determining a progression state for a patient based at least partly on a difference between at least a portion of the autofluorescence spectrum of the imaged tissue and the intensity threshold.

41. (canceled)

42. The method of claim 22, further comprising identifying one or more autofluorescence parameters using the imaged tissue, and storing the one or more autofluorescence parameters in a non-transitory computer-readable medium to monitor the progression and/or regression of the disease state over time.

43. A medical imaging device comprising:
an excitation source that emits excitation light at one or more wavelengths between 400 nm and 850 nm;
a detector configured to detect an autofluorescence signal emitted from lipopigments from tissue being imaged with the device with wavelengths between or equal to 700 nm and 1200 nm;
a computing device that receives the detected autofluorescence signal from the detector, wherein the computing device is configured to continuously determine a disease state of the tissue being imaged in real time.

44-45. (canceled)

46. The medical imaging device of claim 43, wherein the computing device compares the detected autofluorescence signal to an intensity threshold to determine the disease state.

47-48. (canceled)

49. The medical imaging device of claim 43, wherein the computing device is configured to determine a progression state of the disease state based at least partly on an intensity and/or area of the detected autofluorescence signal.

50. (canceled)

51. The medical imaging device of claim 49, wherein the computing device is configured to output the determined disease state, progression state, and detected autofluorescence signal to a display.

52. The medical imaging device of claim 43, wherein the excitation source and the detector are configured to image anatomical structures through intervening tissue.

53. (canceled)

54. The medical imaging device of claim 43, wherein the computing device is configured to identify one or more autofluorescence parameters using the detected autofluorescence signal, and store the one or more autofluorescence parameters in a non-transitory computer-readable medium to monitor the progression and/or regression of the disease state over time.
